(19) United States
(12) Patent Application Publication
(10) Pub. No.: US 2025/0269361 A1
(43) Pub. Date: Aug. 28, 2025

(54) NEW SYNTHESES OF Z-SELECTIVE OLEFIN METATHESIS CATALYSTS

(71) Applicants: Umicore AG & Co. KG, Hanau-Wolfgang (DE); California Institute of Technology, Pasadena, CA (US)

(72) Inventors: Yan XU, Pasadena, CA (US); Adam M. JOHNS, Claremont, CA (US); Robert H. GRUBBS, Pasadena, CA (US); Adrian E. SAMKIAN, La Crescenta, CA (US)

(21) Appl. No.: 17/626,847

(22) PCT Filed: Jul. 7, 2020
(86) PCT No.: PCT/US2020/040954

§ 371 Date: Jan. 13, 2022

Related U.S. Application Data
(60) Provisional application No. 62/875,156, filed on Jul. 17, 2019.

(51) Int. Cl. B01J 31/22; B01J 37/04; C07C 5/22; C07F 15/00

(52) U.S. Cl. CPC B01J 31/2234; B01J 31/223; B01J 31/2273; B01J 31/2291; B01J 31/2295; B01J 37/04; C07C 5/2286; C07F 15/0046; B01J 2231/543; B01J 2531/004; B01J 2531/0288; B01J 2531/821

(57) ABSTRACT

The present disclosure is directed to methods preparing ruthenium compounds, useful for use in metathesis reactions, the product compounds arising from such methods, and the use of such product compounds as catalysts in the metathesis of olefins. In particular, methods take advantage of the use of intermediates of the general formula (C) to obtain the C—H metalated, sterically hindered ruthenium compounds of generally formula (B): Using the disclosed methods, the compounds of the general formula (B) are produced in higher yields, exhibit higher catalytic activity/selectivity, and give rise to more hindered catalysts than are accessible from methods previously known.

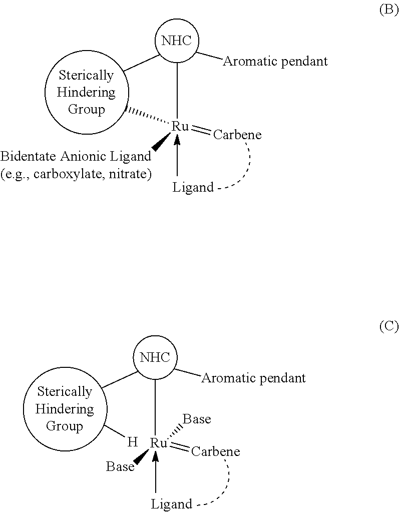

### CROSS REFERENCE TO RELATED APPLICATIONS

This application is a national stage application (under 35 U.S.C. § 371) of PCT/US2020/040954, filed Jul. 7, 2020, which claims benefit of U.S. Patent Application No. 62/875,156, filed Jul. 17, 2019, both of which are incorporated herein by reference in their entirety.

### GOVERNMENT RIGHTS

This invention was made with government support under Grant No. GM031332 awarded by the National Institutes of Health and Grant No. CHE1502616 awarded by the National Science Foundation. The government has certain rights in the invention.

### TECHNICAL FIELD

The present disclosure is directed to methods preparing ruthenium compounds, useful in metathesis reactions, preferably for preparing sterically hindered ruthenium catalysts, for use in metathesis reactions that result in the preferential formation of Z-olefins, the compounds arising from such methods, and the use of such compounds as catalysts in the metathesis of olefins. These compounds can be used in the fields of catalysis, organic synthesis, polymer chemistry, and in industrial applications such as the production of fine chemicals and pharmaceuticals.

### BACKGROUND

Olefin metathesis is a highly valuable synthetic method for carbon-carbon double bond formation. The technology of ruthenium metathesis catalysts has enabled several reaction platforms with broad applications, including ring opening metathesis polymerization (ROMP), ring opening cross metathesis (ROCM), cross metathesis (CM), ring closing metathesis (RCM), and acyclic diene metathesis (ADMET). In most of these applications, internal olefins are formed as reaction products.

Internal olefins are important building blocks in modern drugs, agrochemicals, and functional materials, and are convenient handles for complex target syntheses. The configuration of internal olefins, commonly defined as E or Z, determine the overall configuration of the molecules, thereby influencing their physical, chemical, and biological properties. Given the general difficulty associated with the separation of E/Z olefin mixtures, the selective construction of internal olefins with precise configuration control is highly desirable in applications varying from laboratory researches to industrial productions, especially in terms of minimizing waste and streamlining synthesis.

Conventional olefin metathesis catalysts generally gave mixtures of Z- and E-olefins. In many of the cases. E-olefins are the major products. When the products result from thermodynamic equilibration, the energy difference between the two isomers yields an E-:Z-ratio of from about 5:1 to about 9:1.

The Grubbs group reported in 2011 that the incorporation of a certain type of carbon-chelating N-Heterocyclic Carbene (NHC) ligands gave rise to the Grubbs Z-selective ruthenium olefin metathesis catalysts that catalyze highly Z-selective olefin metathesis (Endo. K.; Grubbs, R. H. Chelated Ruthenium Catalysts for Z-Selective Olefin Metathesis J. Am. Chem. Soc., 2011, 133. 8525-8527), such as Ru-1 and Ru-2 (FIG. 1).

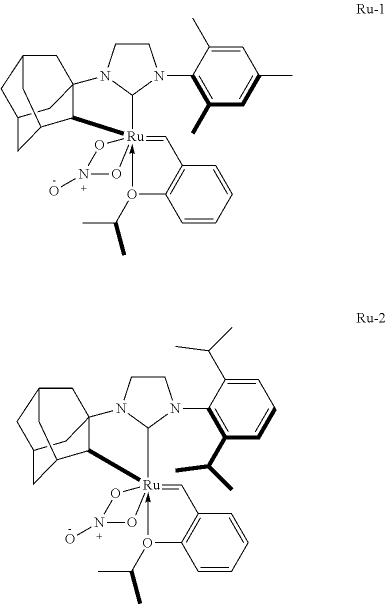

The synthesis of Grubbs Z-selective ruthenium olefin metathesis catalysts with carbon-chelating NHC ligands relied on the intramolecular C—H bond activation of the NHC ligands. This was the key step for the formation of the necessary chelation to achieve appropriate Z-selectivity, and therefore the key step for the entire synthesis.

To achieve this, the Grubbs group developed a method involving the anion ligand substitution between ruthenium dichloride precursors and silver pivalate to form a dipivalate compound (Endo, K.; Grubbs, R. H. Chelated Ruthenium Catalysts for Z-Selective Olefin Metathesis J. Am. Chem. Soc. 2011, 133, 8525-8527). The generated ruthenium dipivalate then underwent simultaneous C—H bond activation to form the key Ru—C bond as well as the desired chelation. However, the use of silver pivalate was extremely expensive, especially on a commercial scale. Further, while this method was effective for the synthesis of compounds such as Ru-1 with a small mesityl group on the NHC (Ar=Mes), this method could not be used to prepare compounds with bulkier pendants, such as Ru-2 with a larger 2,6-diisopropylphenyl group (Ar=Dipp), which were more efficient and selective catalysts in many cases, due to silver-induced decomposition. In 2013, the Grubbs group reported a modified method using sodium pivalate in place of silver pivalates, which gave access to Ru-2 and its related catalysts (Rosebrugh, L. E.; Herbert, M. B.; Marx, V. M.; Keitz, B. K.; Grubbs, R. H. Highly Active Ruthenium Metathesis Catalysts Exhibiting Unprecedented Activity and Z-Selectivity J. Am. Chem. Soc. 2013, 135, 1276-1279); see also U.S. Pat. No. 9,586,981. However, this modified method still required a large quantity of expensive reagent (sodium pivalate, 10 equivalents) and non-trivial, time consuming reaction conditions (40° C., for 4 days), especially for industrial scale-up. More importantly, the overall yield was low (20-30%), which inevitably increased the cost of catalyst production and significantly retarded the commercial application of this type of important Z-selective olefin metathesis catalysts. In addition, given the extreme difficulty in introducing sterically demanding pivalates onto sterically hindered Ru-precursors, Z-selective catalysts bearing NHCs with Ar larger than Dipp, which may possess further improved catalytic activity, have not been successfully synthesized previously (FIGS. 2A-B).

Therefore there is an immediate need for efficient, high-yielding, and ease in scaling up procedures for the synthesis of Grubbs Z-selective ruthenium olefin metathesis catalysts, particularly the C—H bond activation step to form chelation. The present disclosure addresses some of these shortfalls in the prior art.

### SUMMARY

To meet the first need, the inventors have discovered novel syntheses of Grubbs Z-selective ruthenium olefin metathesis catalysts. In particular, the C—H bond activation was achieved in higher yield by using cheaper reagents (e.g. exemplified here with potassium tert-butoxide/lithium pyrrolate and pivalic acid) compared to the existing procedures. See. e.g., FIG. 3.

The present disclosure is directed to methods of preparing compounds, especially sterically hindered compounds, of the general Formula (C)

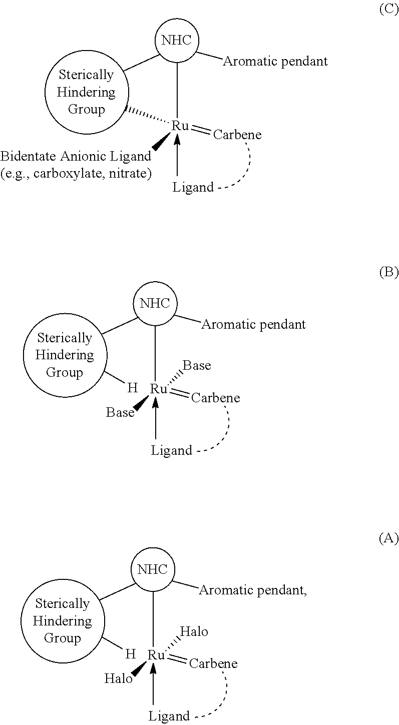

or stereoisomers thereof, the method comprising a series of steps including the step of reacting a compound of the corresponding general Formula (B) with a carboxylic acid to form the compound of general Formula (C), where the bidentate anionic ligand is a carboxylate. The compound of the general Formula (B) may be formed by reacting a dihalo compound of the general corresponding Formula (A) with an anionic base, capable of displacing the halo ligands and coordinating to the ruthenium center as monodentate ligands to form an optionally isolated intermediate. Subsequent reactions can be used to displace the carboxylate ligand with other bidentate anionic ligands, for example nitrate and others. Each of the moieties described as NHC (N-Heterocyclic carbene), Aromatic pendant, Sterically Hindering Group. Base, Bidentate Anionic Ligand. Halo, Carbene, and Ligand are described herein.

Within this general context, certain embodiments include those methods of preparing a compound of Formula (III) or (XIII), the method comprising contacting a compound of Formula (II) or (XII), respectively, or geometric isomer thereof with a carboxylic acid of formula Z—H, wherein the contacting results in the formation of the compound of Formula (III) or (XIII), respectively, or geometric isomer thereof:

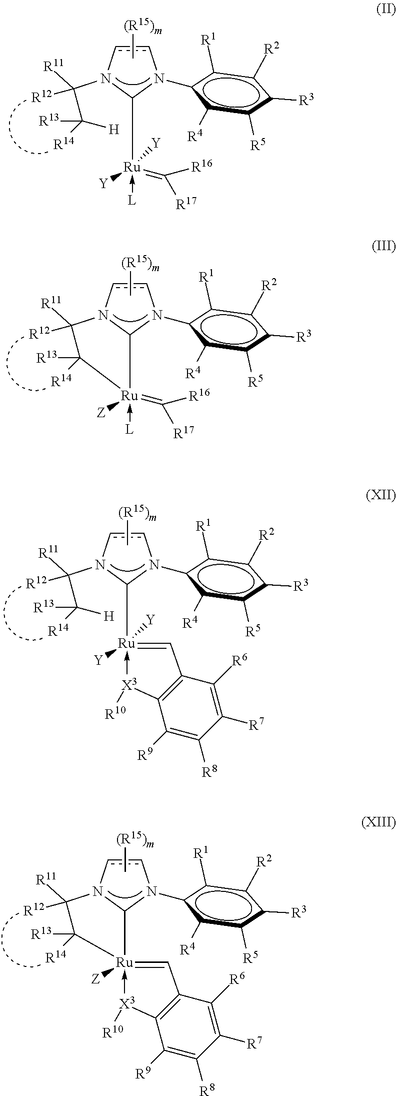

The variables R¹, R², R³, R⁴, R⁵, R⁶, R⁷, R⁸, R⁹, R¹⁰, R¹¹, R¹², R¹³, R¹⁴, R¹⁵, and m define various and optional substituents on these structures. Y is independently a basic anion coordinated as a ligand to the ruthenium center. Z is an optionally substituted carboxylate anion, preferably a tertiary carboxylate anion, coordinated as a ligand to the ruthenium center, L is a donor ligand. X³ is a heteroatom capable of acting as a ligand to the ruthenium center. Each of these variable is defined more explicitly elsewhere herein.

Other embodiments include methods comprising contacting a compound of Formula (I) or (XI), or geometric isomer thereof, with a salt of formula [M⁺][Y⁻], wherein the contacting results in the formation of the compound of Formula (II) or (XII), respectively, or geometric isomer thereof:

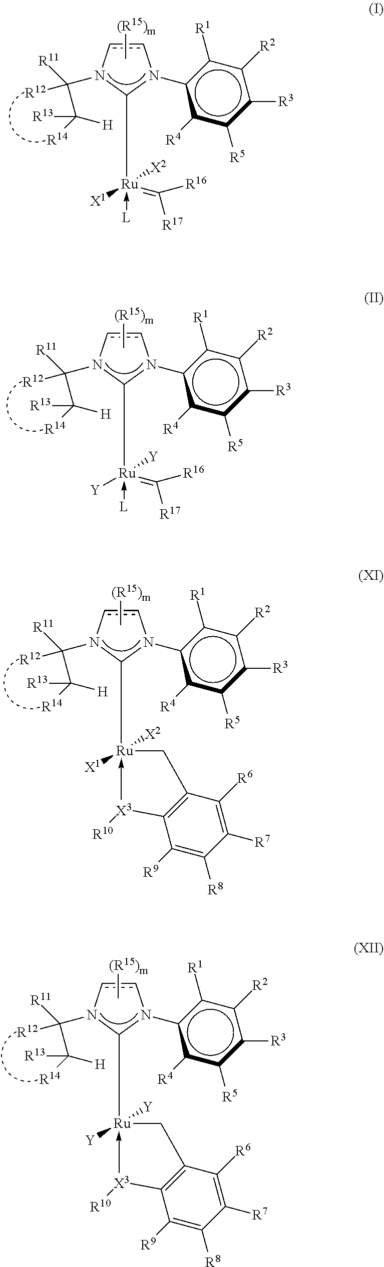

wherein:
   X¹ and X² are independently halo (e.g., chloro, bromo, or iodo);
   M⁺ is a monocation equivalent cation, as set forth herein.

In some aspects of this embodiment, the methods may be conducted independently or in tandem with the earlier described methods involving the conversion of the compounds of Formula (II) to (III) or of Formula (XII) to (XIII).

In aspects or embodiments. M⁺ is defined in terms of alkali metal cation, an ammonium cation, or other nitrogen-based cation though other cation options (e.g., di- or trivalent cations) are also considered.

Still other embodiments include those methods comprising reacting the compound of formula (III) or (XIII), or geometric isomer thereof, with a trifluoroacetate, nitrate, nitrite, oxalate, phosphate, sulfite, or sulfonate, or other anion of a strong acid (H-Q), capable of coordinating the ruthenium as a bidentate ligand, to form a compound of formula (IV) or (XIV), respectively, or geometric isomer thereof:

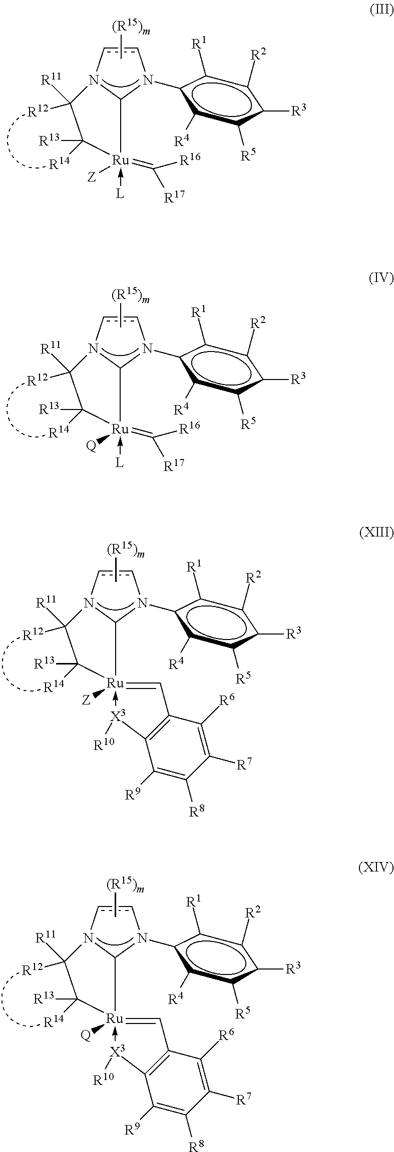

wherein Q is a coordinated trifluoroacetate, nitrate, nitrite, oxalate, phosphate, sulfite, sulfonate, or other anion of a strong acid (H-Q), capable of coordinating the ruthenium as a bidentate ligand. Typically these anions are coordinated to the ruthenium as a bidentate ligand. Nitrate appears to be preferred based on the reactivities of such complexes. Ammonium salts of these anions appear to be suitable sorts of these anions. In particular, ammonium nitrate works well in this context.

In some aspects of this embodiment, the methods may be conducted independently or in tandem with the earlier described methods involving the conversion of the compounds of Formula (I) to (II), (II) to (III) or of Formula (XI) to (XII), (XII) to (XIII). Where conducted in tandem, the methods may be conducted without the isolation of the compound of Formula (II) or (XII).

Various aspects of these preceding embodiments include those setting forth specific options for the several variables that define the structures involved in the methods.

These include those that set forth:
(a) specific favored substituent patterns on the Aromatic pendant, including bulky alkyl substituents and optionally substituted fused ring systems, both in terms of positive recitations and exclusions; and
(b) preferences for some of the substituents, including R¹⁰ (and/or R^{10A}) and/or the Sterically Hindering Group include linear, branched, cyclic, or polycyclic alkyl. In some aspects, isopropyl is excluded from the definition of R¹⁰ and/or R^{10A}.

Still other embodiments include those ruthenium compounds that are accessible from the methods set forth herein, including those ruthenium compounds of Formula (XX-A), (XX-B), (XX-C) or (XX-D):

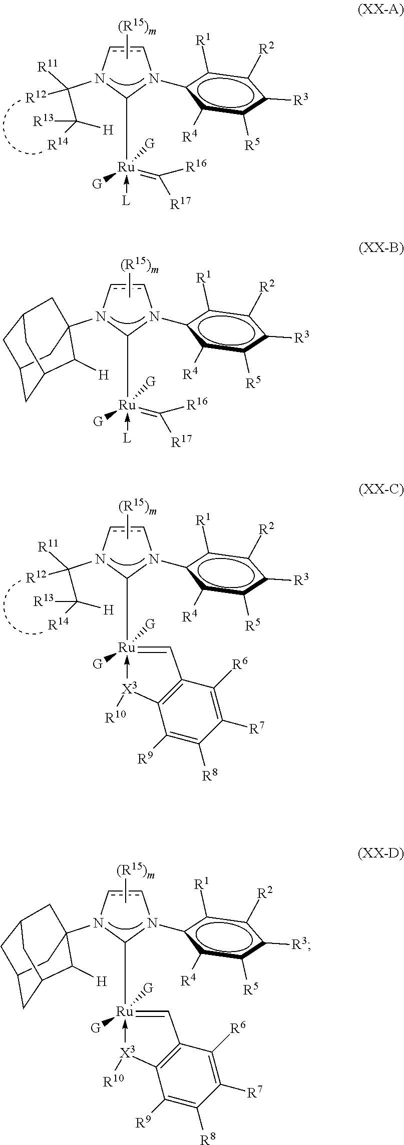

wherein the variables R¹, R², R³, R⁴, R⁵, R⁶, R⁷, R⁸, R⁹, R¹⁰, R¹¹, R¹², R¹³, R¹⁴, R¹⁵, and m define the various and optional substituents on these structures. G includes variables also associated with X¹, X², and Y coordinated as ligands to the ruthenium center, L is a donor ligand. X³ is a heteroatom capable of acting as a ligand to the ruthenium center. Each of these variables is defined more explicitly elsewhere herein. These compounds include those previously not known with respect to the definitions of G, as well as those compounds previously inaccessible and unknown, primarily, but not exclusively because of the bulk of their ligands.

Still other embodiments include those ruthenium compounds that are accessible from the methods set forth herein, including those ruthenium compounds of Formula (Y-A), (Y-B), (Y-C), or (Y-D):

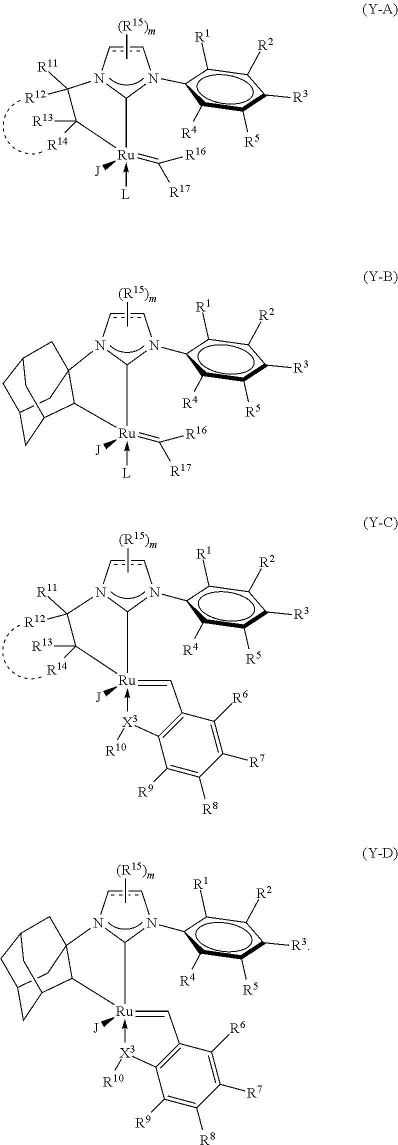

wherein the variables R¹, R², R³, R⁴, R⁵, R⁶, R⁷, R⁸, R⁹, R¹⁰, R¹¹, R¹², R¹³, R¹⁴, R¹⁵, and m define various and optional substituents on these structures. J includes but is not limited to those variables also associated with X¹, X², Y, Q, and Z coordinated as ligands to the ruthenium center. L is a donor ligand. X³ is a heteroatom capable of acting as a ligand to the ruthenium center. Each of these variables is defined more explicitly elsewhere herein. These compounds include those previously not known with respect to the definitions of J, as well as those compounds previously inaccessible and unknown, primarily, but not exclusively because of the bulk of their ligands.

Still other embodiments include the use of any of the compounds set forth herein as catalysts in metathesis reactions. In some such embodiments, the processes comprising contacting at least one alkene, alkyne, or enyne with any of the ruthenium compounds set forth herein, the contacting resulting in a metathesis, for example:
(a) a cross metathesis (CM) of two different alkenes, or an alkene, alkyne, and/or enyne;
(b) a ring closing metathesis of a diene (RCM) leading to the formation of cycloalkene ring;
(c) a homodimerization of two olefins
(d) an acyclic diene metathesis (ADMET) leading to the formation of an oligomer and/or a polymer;
(e) a ring opening cross metathesis (ROCM) leading to an acyclic diene;
(f) a ring opening metathesis polymerization (ROMP) of a cycloalkene leading to a polymer; or
(g) ethenolysis of an E/Z isomer mixture of olefins to provide an enrichment of the E-olefin olefin content of the mixture.

In certain preferred embodiments (e.g., (a) to (f)), the catalysts provide that the favored product of the metathesis reaction(s) is predominantly a Z-isomer olefin. In the case of (g), the favored reactant is the Z-isomer.

### BRIEF DESCRIPTION OF THE DRAWINGS

The present application is further understood when read in conjunction with the appended drawings. For illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1A illustrates general scheme for Z-selective olefin metathesis and commercially available Grubbs Z-catalysts. FIG. 1B depicts one possible representation of the steric interactions leading to the favored Z-selective metathesis pathway

FIGS. 2A-B illustrate prior art attempts to form catalysts through anion exchange directly with carboxylate anions, especially troublesome for large NHCs, Reactions were sluggish and low yielding with bulkier Ar groups (i.e., Ar≥Dipp). When Ar=Mes, both NaOPiv and AgOPiv gave high yields. When Ar=Dipp, NaOPiv gave 20% yields (10 equiv, 4 d, 40° C.) and AgOPiv decomposed (see FIG. 2B). When Ar≥Dipp, product was inaccessible.

FIG. 3 illustrates an exemplification of the present disclosure.

FIGS. 4A-C illustrate various of the generic/exemplary method steps of the present disclosure.

FIG. 5 is a representative ¹H-NMR spectrum of crude Ru-4.

FIG. 6 is a representative ¹H-NMR spectrum of Ru-5.

FIG. 7 illustrates is a representative ¹H-NMR spectrum of Ru-7.

FIG. 8 is a representative ¹H-NMR spectrum of Ru-8.

FIG. 9 is a representative ¹H-NMR spectrum of Ru-12.

FIG. 10 provides examples of (Z)-α,β-unsaturated amides in bioactive compounds and as building blocks.

FIG. 11 provides methods and limitations in the prior art for constructing (Z)-α,β-unsaturated amides.

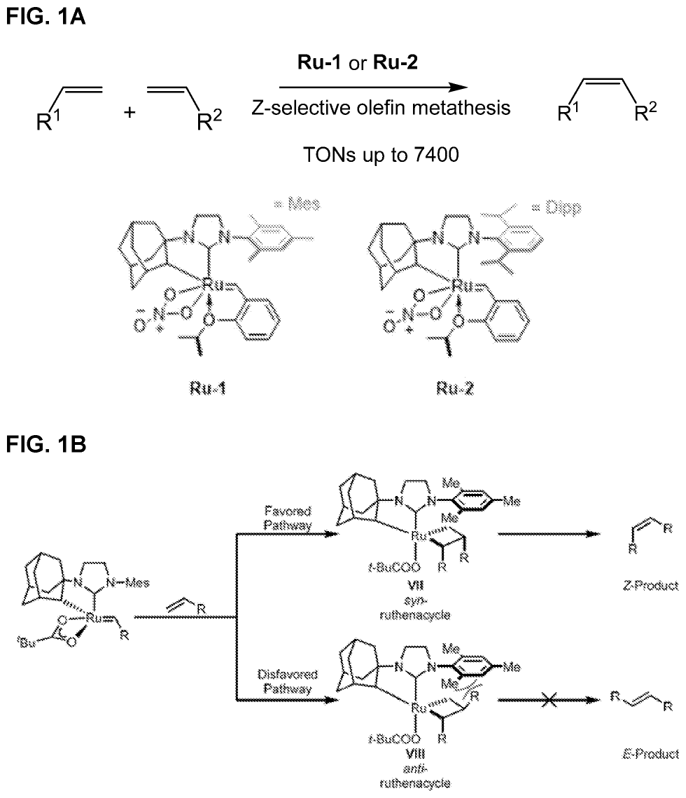

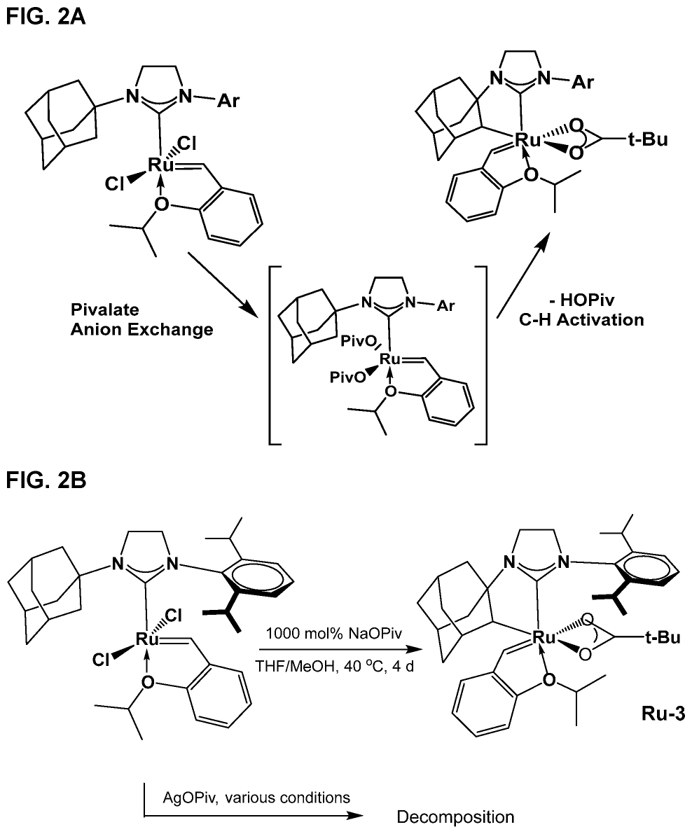

### DETAILED DESCRIPTION OF ILLUSTRATIVE EMBODIMENTS

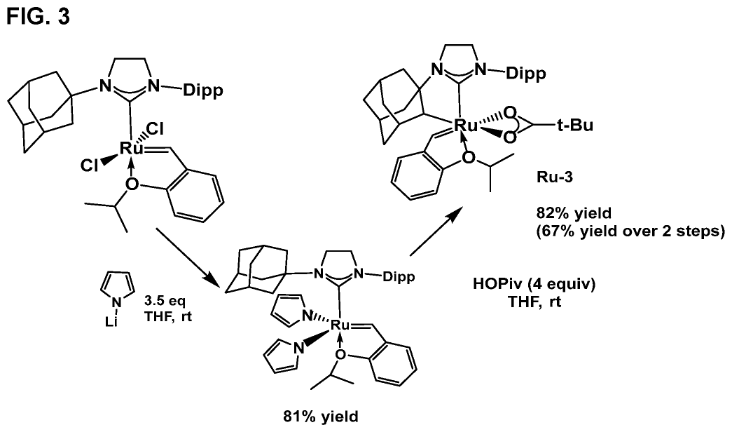

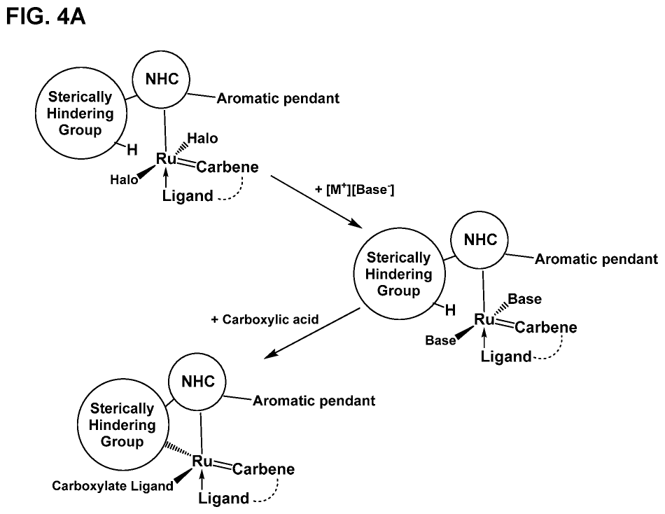

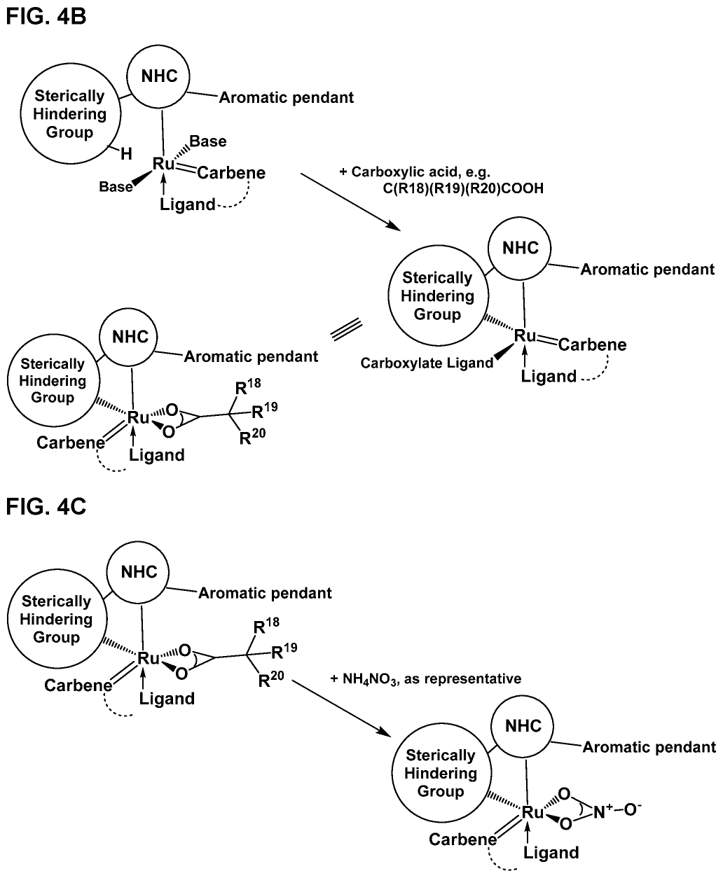

The present disclosure is directed to methods for preparing ruthenium catalysts, including Grubbs Z-selective ruthenium olefin metathesis catalysts, that overcome the difficulties in the introduction of pivalate and other carboxylates and the associated C—H bond activation steps. Previously, the ruthenium pivalate intermediates were synthesized through a direct halide-pivalate anion exchange, which proved to be troublesome with sterically demanding NHC ligands. Herein, a new approach is described, which in one aspect includes the use of ruthenium halide precursors react with a strong base to generate ruthenium base complexes, and then be treated with pivalic acid to yield ruthenium bis-pivalates which undergoes simultaneous C—H activation. With the proper choice of the base, the first step was shown to proceed smoothly, under mild conditions with high yields, even in the presence of sterically demanding NHCs (e.g. where Ar is at least as sterically challenging as with Dipp). The acid-base quenching between the ruthenium base complexes and the pivalic acid provided a strong thermodynamic driving force for the second step, allowing it to occur with high yields. Besides the high overall yields, this new approach further features the use of cheap reagents (e.g. potassium tert-butoxide and lithium pyrrolate as the bases, see vide infra, and pivalic acid) and industrial-friendly reaction conditions (e.g. at near room temperature). As one representative example, while the previous approaches were only able to produce Ru-3 in 20-30% yield (precursor to the commercially available Ru-2), this new approach resulted in yields of about 70% yield from the identical ruthenium dichloride starting material by using cheap lithium pyrrolate and pivalic acid as the reagents. In addition, these methods further gives access to Z-selective ruthenium olefin metathesis catalysts bearing NHCs with Ar larger than Dipp. Catalysts based on these more sterically demanding ligands provide unique and more selective catalytic activity compared to the existing Z-selective olefin metathesis catalysts in certain applications, as described herein (see FIGS. 3, 4A/B).

As set forth elsewhere herein, the present disclosure is directed to methods of preparing compounds, especially sterically hindered compounds, of the general Formula (C)

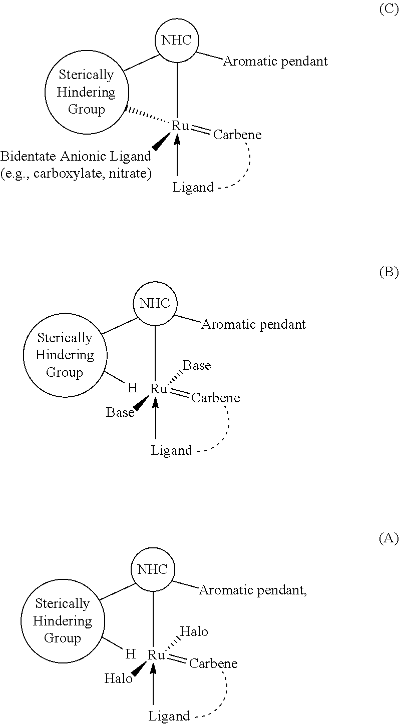

or stereoisomers or geometric isomers thereof, the method comprising a series of steps including the step of reacting a compound of the corresponding general Formula (B) with a carboxylic acid to form the compound of general Formula (C), where the bidentate anionic ligand is a carboxylate. The compound of the general Formula (B) may be formed by reacting a dihalo compound of the general corresponding Formula (A) with an anionic base, capable of displacing the halo ligands and coordinating to the ruthenium center as monodentate ligands to form an optionally isolated intermediate. Subsequent reactions can be used to displace the carboxylate ligand with other bidentate anionic ligands, for example nitrate and others.

Within this general context, there is a common set of descriptors presented for the methods and compounds of the present disclosure.

For example, the compounds set forth above as general formula (A) are more specifically described in terms of compounds of formulae (I) or (XI):

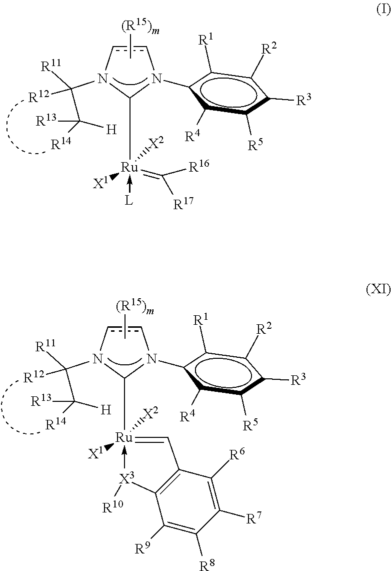

For example, the compounds set forth above as general formula (B) are more specifically described in terms of compounds of formulae (H) or (XII):

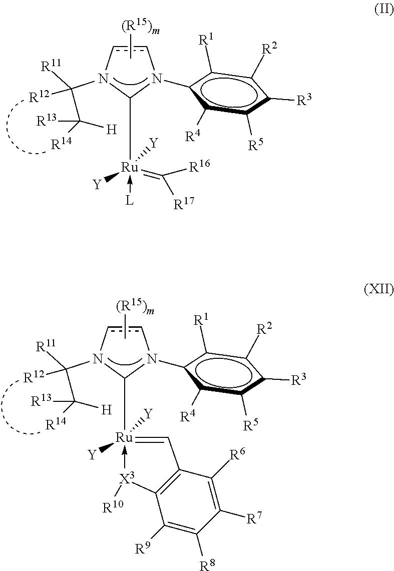

For example, the compounds set forth above as general formula (B) are more specifically described in terms of compounds of formulae (III), (XIII), (IV), and (XIV)

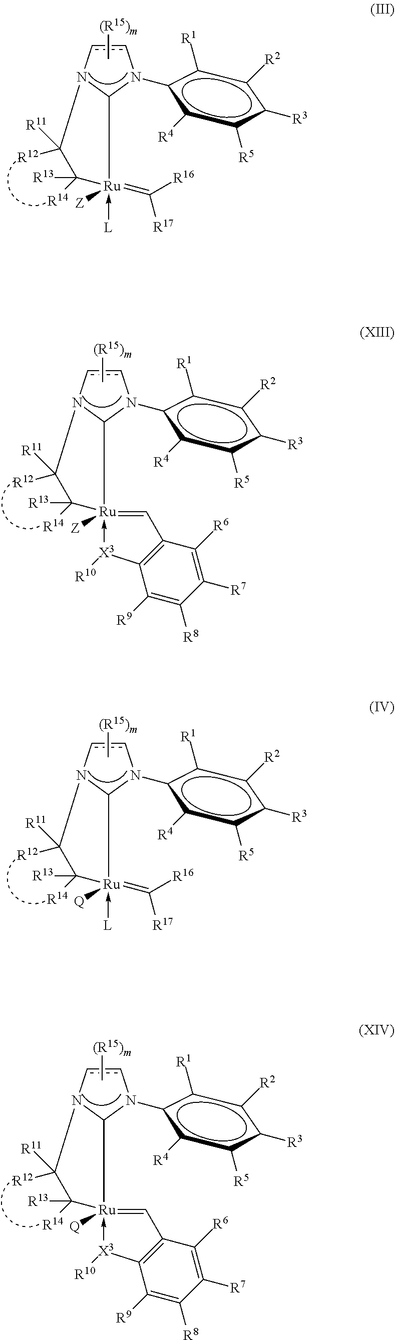

Within each of these contexts, then, Halo can be represented by X¹ and X², the anionic Base can be represented by Y, the carboxylate ligand can be represented by Z, the other bidentate anionic ligands can be represented by Q, the so-called NHCs include the structures of

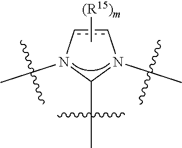

the Aromatic pendants include the structures

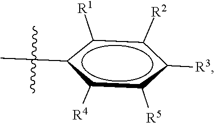

the Sterically Hindered Groups include the structures

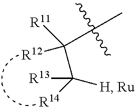

and the Ruthenium Carbene/Ligand include the structures:

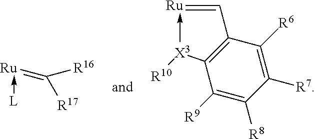

Accordingly, some embodiments include methods of preparing a compound of Formula (III) or (XIII), each method comprising contacting a compound of Formula (II) or (XII), respectively, or geometric isomer thereof with a carboxylic acid of formula Z—H, wherein the contacting results in the formation of the compound of Formula (IV) or (XIV), respectively, or geometric isomer thereof.

Accordingly, some other embodiments include methods of preparing a compound of Formula (II) or (XII), each method comprising contacting a compound of Formula (I) or (XI), or geometric isomer thereof, with a salt of formula [M⁺][Y⁻], wherein the contacting results in the formation of the compound of Formula (II) or (XII), respectively.

Accordingly, some other embodiments include methods of preparing a compound of Formula (IV) or (XIV), each method comprising reacting the compound of formula (III) or (XIII), or geometric isomer thereof, with a trifluoroacetate, nitrate, nitrite, oxalate, phosphate, sulfite, or sulfonate or other anion of a strong acid (H-Q), capable of coordinating the ruthenium as a bidentate ligand, to form a compound of formula (IV) or (XIV), respectively, or geometric isomer thereof.

The conditions useful for affecting these transitions (times, temperatures, solvents, and nature of the non-oxidative environment) are consistent with those normally considered by persons skilled in the art of such chemistries, but for the sake of completeness, these are set forth elsewhere herein.

While the structures set forth herein are generally represented in one geometry or stereochemistry, the descriptions and claims are not intended to be limited to these stereochemistries and the compounds themselves may be represent as alternative isomers.

Within these methods, each of the substituents R¹, R², R³, R⁴, R⁵, R⁶, R⁷, R⁸, R⁹, R¹⁰ (and R^{10A}), R¹¹, R¹², R¹³, R¹⁴, R¹⁵, R¹⁶, R¹⁷, m, X¹, X², X³, L, Y, Z, and Q are, in most cases, defined as follows:
R¹, R², R³, R⁴, R⁵, R⁶, R⁷, R⁸, and R⁹ independently are or include hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups; and/or one or more of R¹ and R² or R² and R³ or R³ and R⁵ or R⁴ and R⁵ or R⁶ and R⁷ or R⁷ and R⁸ or R⁸ and R⁹, together with the carbons to which they are bound, form at least one optionally substituted 5- to 7-membered ring structure;
R¹¹, R¹², R¹³, and R¹⁴ independently are or include hydrogen, halogen, optionally substituted alkyl, optionally substituted alkenyl, optionally substituted alkoxyl, optionally substituted aryl, optionally substituted heteroaryl; and/or one or more pairs of R¹¹, R¹², R¹³, and R¹⁴ are independently optionally linked together to form a cyclic or polycyclic structure;
R¹⁵ independently is or includes an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure (e.g., comprising linked alkylenes or ethers):

m is 0, 1, 2, 3, or 4;
R¹⁶ and R¹⁷ independently are or include hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy;
X¹ and X² are independently chloro, bromo, or iodo;
X³ is —O—, —S—, or —N(R^{10A})—;
R¹⁰ and R^{10A} independently are or include optionally substituted linear, branched, cyclic, or polycyclic alkyl, optionally substituted aryl, optionally substituted heteroaryl;
the designation [symbol] refers to the presence of an optional double bond;
L is a donor ligand, optionally a neutral electron donor ligand, optionally a neutral electron donor solvent or an electron donor ligand linked to R¹⁷.
m is 0, 1, 2, 3, or 4;
R¹⁵ independently is or includes an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure (e.g., comprising linked alkylenes or ethers);
Y independently is or includes a basic anion coordinated as a ligand to the ruthenium center;

[M⁺][Y⁻] is a salt of Y⁻, wherein M⁺ is a monocation equivalent cation, preferably but not necessarily an alkali metal, optionally substituted ammonium, or pyridinium cation.
Z is a carboxylate, optionally of formula C(R¹⁸)(R¹⁹)(R²⁰)C(═O)O⁻;
R¹⁸, R¹⁹, and R²⁰ independently are or include hydrogen, optionally substituted alkyl, optionally substituted alkenyl, optionally substituted aryl, or optionally substituted heteroaryl, provided that not all of R¹⁸, R¹⁹, and R²⁰ are hydrogen; and/or two or three of R¹⁸, R¹⁹, and R²⁰ are linked together to form a cyclic or polycyclic structure; and
Q is trifluoroacetate, nitrate, nitrite, oxalate, phosphate, sulfite, or sulfonate or other anion of a strong acid (H-Q), capable of coordinating the ruthenium as a bidentate ligand. In preferred embodiments, Q is delivered as its ammonium, NH₄⁺, salt.

Additional aspects of these definitions are also provided elsewhere herein, for example, additional substituents (including those described in terms of “Fn”), exclusions/provisos, and exemplary structures.

For example, in the case of the NHCs (N-heterocyclic carbenes) and aromatic pendants therefrom, though it should be appreciated that the disclosure is not necessarily limited to these structures, additional embodiments also embrace those compounds, for example, where the NHC ring is a 6-membered ring optionally substituted with one or more R¹⁵ substituents.

Also, the representations of the NHC core and its associated descriptors:

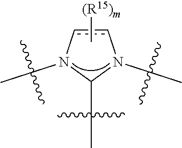

includes at least the structures where the ring independently and alternatively contains a single or a double bond (as designated by [symbol] ). Within the context of (R¹⁵)ₘ, the difference also manifesting itself with different potential positions and distributions of the R¹⁵ groups:

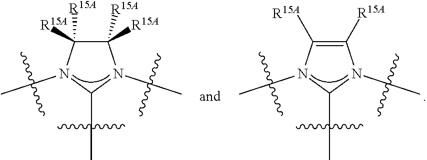

where R¹⁵ is redefined as R^{15A} (if only to reflect that the absence of a non-hydrogen substituent is H) to include the additional presence of H.

For example, in the case of the Aromatic pendant, in addition to the description as

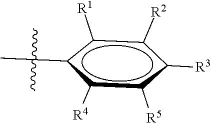

and the recited descriptions for R¹, R², R³, R⁴, and R⁵, which include the embodiments that R¹ and R² or R² and R³ or R³ and R⁵ or R⁴ and R⁵, together with the carbons to which they are bound, form at least one optionally substituted 5- to 7-membered ring structure (for example, optionally substituted aryl, heteroaryl, cyclic alkylene, cyclic alkenylene, or heterocyclic moieties fused to the Aromatic pendent group), in certain embodiments, the Aromatic pendant is itself heteroaryl moiety, either fused to or in substitution for, the phenyl group as shown in the Formulae (I), (XI), (II), (XII), (III), (XIII), (IV), and (XIV).

For example, in the context of the Sterically Hindering Group pendant to the NHCs and, in some cases, bonded to the ruthenium center, is sometimes presented in terms of the structures:

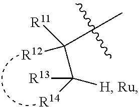

in addition to the optional additional functional groups embraced by the designation as “Fn” set forth elsewhere herein, the description that any two or more of R¹¹, R¹², R¹³, and R¹⁴ can be linked to form cyclic or polycyclic structures refers to the fact that this group can include, in addition to optionally substituted adamantane can also include other optionally substituted bicyclic or polycyclic structure, such as optionally alkyl substituted bicyclopentanes, bicyclohexanes, bicycloheptanes, and bicyclooctanes or bulky aromatic structures, for example:

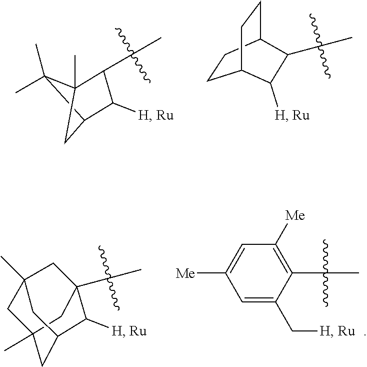

For example, Ruthenium Carbene/Ligand described in term of the structures:

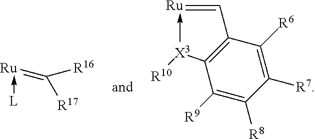

also embrace those embodiments where the 6-membered carbocyclic aryl group is replaced by an optionally substituted heteroaryl group, for example (but not limited to pyridine). A more complete non-limiting listing of the heteroaryl groups contemplated by this description are set forth elsewhere herein.

It should be appreciated that within the full context of the definitions of L, R¹⁶, and R¹⁷, the structures embracing these terms represent genera of, and embrace, the structures defined in terms of X³, R⁶, R⁷, R⁸, R⁹, and R¹⁰. To the extent that the genus is broader than the species structures, other species include those structures encompassing the difference between the two groups; e.g., those compounds included within the genus embracing the L, R¹⁶, and R¹⁷ moieties but not in the species embraced by the X³, R⁶, R⁷, R⁸, R⁹, and R¹⁰ moieties. For example, in certain embodiments, these carbenes independently include Fischer-type carbenes (in which R¹⁶ or R¹⁷ comprises an optionally substituted alkoxy, aryloxy, or heteroaryloxy pendant) as well as the more active “Schrock-type” congeners. In other embodiments, the compound may comprise structures in which the monodentate ligand L is absent or displaced by a heterocyclic moiety linked to the carbene group by an alkyl or ether linkage.

For example, L is defined in terms of a neutral electron donor ligand, optionally a neutral electron donor solvent or an electron donor ligand linked to R¹⁷. Also included in the context of the compounds of the present disclosure are those compounds and structures where L is absent; i.e., this group is neither provided by a carbene pendent or separately present and the ruthenium center is further coordinatively unsaturated. Where present, L may include any one or more of the neutral electron donor ligands set forth elsewhere herein.

Throughout this disclosure Y is described as independently a basic anion coordinated as a ligand to the ruthenium center (when associated as a ligand) or as an anion, Y⁻, when defined in terms of a salt [M⁺][Y⁻]. In certain characterizations, Y may be seen as relatively weakly nucleophilic that does not decompose the ruthenium carbene but sufficiently nucleophilic to displace halide from a corresponding Ru center (as set forth elsewhere herein) and sufficiently basic to be protonated by a carboxylic acid. The specific parameters of these reactivities are set forth elsewhere herein. In preferred embodiments. [M⁺][Y⁻] is potassium tert-butoxide or lithium pyrrolate, though other such basic salts are useful for this purpose.

In some embodiments, Y is independently a hydroxide. C₁₋₁₂ alkoxide, an amidate anion, substituted or unsubstituted phenoxide, substituted or unsubstituted pyrrolate, substituted or unsubstituted indolate, substituted or unsubstituted isoindolate, or substituted or unsubstituted imidazolate. Some preferred exemplars of these categories are set forth elsewhere herein.

Throughout this disclosure, Z is a carboxylate and Z—H is the corresponding carboxylic acid. In this disclosure, Z is generally described in terms of the formula C(R¹⁸)(R¹⁹)(R²⁰)COOH, where R¹⁸, R¹⁹, and R²⁰ are provided elsewhere herein. In preferred embodiments, Z is a carboxylate of at least a secondary carbon group carbon group. Bulkier, tertiary carboxylates appear to be preferred. Representative exemplars of these carboxylates are set forth elsewhere herein.

In some embodiment, the contacting with the carboxylic is accompanied by the additional use of the corresponding carboxylate, preferably an alkali metal carboxylate.

Still other embodiments include those ruthenium compounds that are accessible from the methods set forth herein, including those ruthenium compounds of Formula (XX-A), (XX-B), (XX-C) or (XX-D):

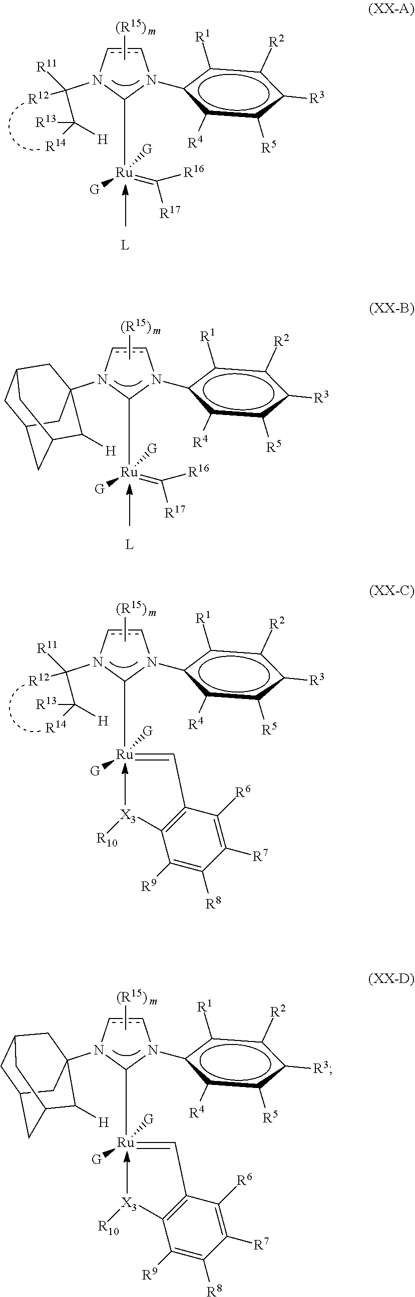

wherein the variables R¹, R², R³, R⁴, R⁵, R⁶, R⁷, R⁸, R⁹, R¹⁰, R¹¹, R¹², R¹³, R¹⁴, R¹⁵, and m are consistent with the previous or elsewhere described descriptions for these terms. G includes variables also associated with X¹, X², and Y coordinated as ligands to the ruthenium center. These compounds include those previously not known with respect to the definitions of G, as well as those compounds previously inaccessible and unknown, primarily, but not exclusively because of the bulk of their ligands.

Still other embodiments include those ruthenium compounds that are accessible from the methods set forth herein, including those ruthenium compounds of Formula (Y-A), (Y-B), (Y-C), or (Y-D):

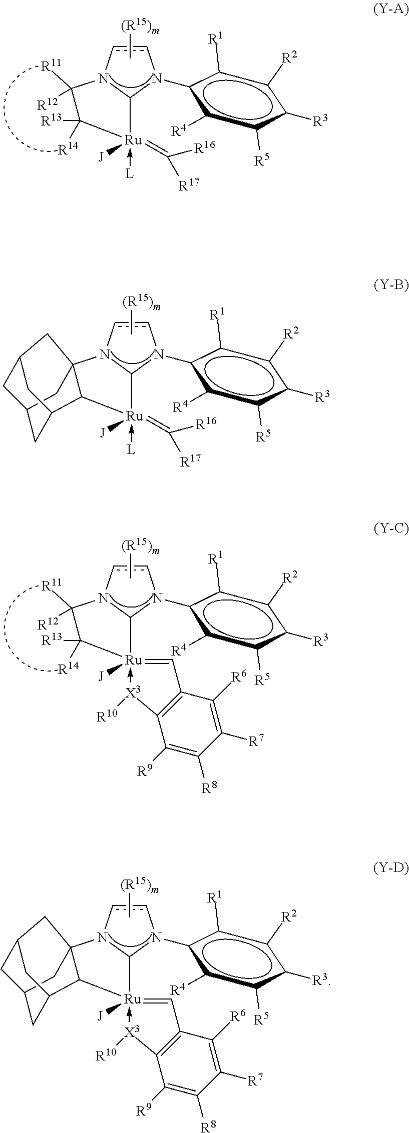

wherein the variables R¹, R², R³, R⁴, R⁵, R⁶, R⁷, R⁸, R⁹, R¹⁰, R¹¹, R¹², R¹³, R¹⁴, R^{1S}, and m define are consistent with the previous descriptions for these terms. J includes but is not limited to those variables also associated with X¹, X², Y, Q, and Z coordinated as ligands to the ruthenium center. These compounds include those previously not known with respect to the definitions of J, as well as those compounds previously inaccessible and unknown, primarily, but not exclusively because of the bulk of their ligands

Still other embodiments include the use of any of the compounds set forth herein as catalysts in metathesis reactions. In some such embodiments, the processes comprising contacting at least one alkene, alkyne, or enyne with any of the ruthenium compound as set forth herein, the contacting resulting in a metathesis, for example:
(a) a cross metathesis (CM) of two different alkenes, or an alkene, alkyne, and/or enyne, for example:

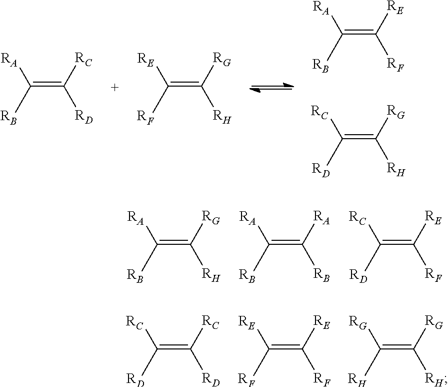

(b) a ring closing metathesis of a diene (RCM) leading to the formation of cycloalkene ring, for example

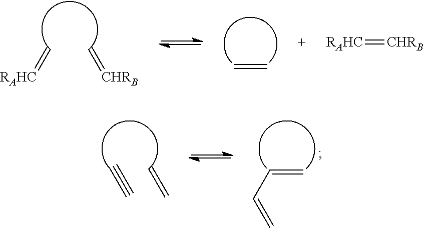

(c) a homodimerization of two olefins, for example:

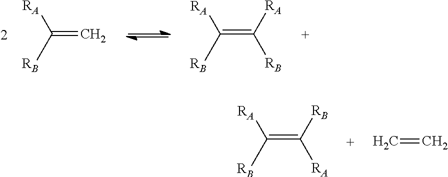

(d) an acyclic diene metathesis (ADMET) leading to the formation of an oligomer and/or a polymer, for example

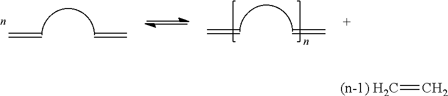

(e) a ring opening cross metathesis (ROCM) leading to an acyclic diene;

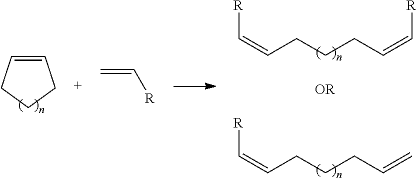

(f) a ring opening metathesis polymerization (ROMP) of a cycloalkene leading to a polymer, for example

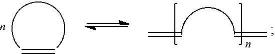

(g) ethenolysis of an E/Z isomer mixture of olefins to provide an enrichment of the E-olefin content of the mixture, for example

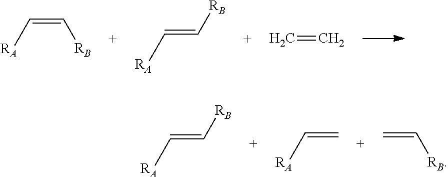

With respect to this last-mentioned process, many methods exist for preparing disubstituted E-olefins, such as the Homer-Wadsworth-Emmons reaction and olefin cross-metathesis. While these reactions have proven to be highly reliable and practical, the undesired Z-isomer is often difficult to separate by chromatography or distillation. In the present disclosure, the use of the presently described ruthenium compounds are useful for providing practically pure, or at least substantially enriched E-olefins from an E/Z mixture through the selective ethenolysis of the Z-isomer. The difference between the use of the present and conventional olefin metathesis catalysts is not just steric: (a) the Z-selective ethenolysis is not a Z-to-E isomerization reaction—instead, it preferentially transforms Z-olefins to terminal olefins (not E-olefin), and preferentially leaves E-olefins untouched. (b) these more hindered catalysts can give >95% (or even >98%) E-isomer, which is through kinetic-control by the catalyst. The conventional Z-to-E isomerization gives 5:1˜10:1 E-/Z- mixtures in most cases, which are dictated by the inherent thermodynamic difference between the two isomers.

In certain preferred embodiments, (e.g., (a) to (f)), the catalysts provide that the favored product of the metathesis reaction(s) is predominantly a Z-isomer olefin. In the case of (g), the favored reactant is the Z-isomer, leading to its preferential removal from the mixture.

### Terms

The present disclosure may be understood more readily by reference to the following description taken in connection with the accompanying Figures and Examples, all of which form a part of this disclosure. It is to be understood that this disclosure is not limited to the specific products, methods, conditions or parameters described or shown herein, and that the terminology used herein is for the purpose of describing particular embodiments by way of example only and is not intended to be limiting of any claimed disclosure. Similarly, unless specifically otherwise stated, any description as to a possible mechanism or mode of action or reason for improvement is meant to be illustrative only, and any invention disclosed herein is not to be constrained by the correctness or incorrectness of any such suggested mechanism or mode of action or reason for improvement. Throughout this text, it is recognized that the descriptions refer to compositions and methods of making and using said compositions. That is, where the disclosure describes or claims a feature or embodiment associated with a composition or a method of making or using a composition, it is appreciated that such a description or claim is intended to extend these features or embodiment to embodiments in each of these contexts (i.e., compositions, methods of making, and methods of using).

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the disclosure which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the disclosure that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements or use of a “negative’ limitation. Finally, while an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of” and “consisting essentially of.” For those embodiments provided in terms of “consisting essentially of,” the basic and novel characteristic(s) is the facile operability of the methods (or the systems used in such methods), under conditions comparable to those recited.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Throughout this specification, words are to be afforded their normal meaning, as would be understood by those skilled in the relevant art. Unless defined otherwise, all technical and scientific terms used herein have the same meaning as commonly understood by one of ordinary skill in the art to which any invention disclosed herein belongs. Although any methods and materials similar or equivalent to those described herein can also be used in the practice or testing of the present inventions disclosed, representative illustrative methods and materials are described herein.

The term “alkyl” as used herein refers to a linear, branched, cyclic, bicyclic, or polycyclic saturated hydrocarbon group (and mention of “alkyl” generally embraces each of these as separate embodiments), typically although not necessarily containing 1 to about 24 carbon atoms, preferably 1 to about 12 carbon atoms, such as methyl, ethyl, n-propyl, isopropyl, n-butyl, isobutyl, t-butyl, pentyl, hexyl, heptyl, octyl, decyl, and the like, as well as cycloalkyl groups such as cyclopropyl, cyclobutyl, cyclopentyl, cyclohexyl, cycloheptyl, and the like. Reference to branched alkyls, includes for example, 2-propyl (isopropyl), s-butyl, t-butyl, and isobutyl, 2-pentyl, 3-pentyl, 2-methylbutyl, 3-methylbutyl, neopentyl, 2-hexyl, 3-hexyl, 4 hexyl, 2-heptyl, 3-heptyl, 4 heptyl, and so forth. Bicyclic or polycyclic alkanes include, for example, bicyclopentanes, bicyclohexanes, bicycloheptanes, bicyclooctanes, and adamantanes. Reference to “alkyl” also embraces each of these chain lengths as a separate embodiment.

A reference to a “linear, branched, cyclic, bicyclic, or polycyclic C₃₋₂₄alkyl” is also intended to be interpreted as an alkyl having a minimum number of carbons necessary to provide for the respective alkyl type up to 24 carbons (e.g., branched C₃₋₂₄alkyl, cyclic C₃₋₂₄alkyl, bicyclic C₆₋₂₄alkyl, or polycyclic C₆₋₂₄alkyl). Generally, although again not necessarily, alkyl groups herein contain 1 to about 12 carbon atoms. The term “lower alkyl.” embraces as a separate sub-set embodiment of the term “alkyl,” intends an alkyl group of 1 to 6 carbon atoms. The specific term “cycloalkyl,” also embraced as a separate sub-set embodiment of the term “alkyl,” intends a cyclic alkyl group, typically having 3 to 8, preferably 6 or 7, carbon atoms. The term “substituted alkyl” refers to alkyl groups substituted with one or more substituent groups, as set forth elsewhere herein, and the terms “heteroatom-containing alkyl” and “heteroalkyl” refer to alkyl groups in which at least one carbon atom is replaced with a heteroatom within the carbon chain. Examples of the latter include alkyl ethers, alkyl polyethers (including polyglycols), alkyl thiol ethers, and secondary or tertiary alkyl amines. Generally, and if not otherwise indicated, the terms “alkyl” and “lower alkyl” include linear, branched, cyclic, unsubstituted, substituted, and/or heteroatom-containing alkyl and lower alkyl groups, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, pentenyl, hexenyl, heptenyl, octenyl, decenyl, tetradecenyl, hexadecenyl, eicosenyl, tetracosenyl, and the like. Preferred alkenyl groups herein contain 2 to about 12 carbon atoms. The term “lower alkenyl,” embraced as a separate sub-set embodiment of the term “alkenyl,” intends an alkenyl group of 2 to 6 carbon atoms, and the specific term “cycloalkenyl.” embraced as a separate sub-set embodiment of the term “alkenyl,” intends a cyclic alkenyl group, preferably having 5 to 8 carbon atoms. The term “substituted alkenyl” refers to alkenyl groups substituted with one or more substituent groups, and the terms “heteroatom-containing alkenyl” and “heteroalkenyl” refer to alkenyl groups in which at least one carbon atom within the carbon chain is replaced with a heteroatom. Examples of the latter include alkenyl ethers, alkenyl polyethers (including polyglycols), alkenyl thiol ethers, and secondary or tertiary alkenyl amines. If not otherwise indicated, the terms “alkenyl” and “lower alkenyl” include linear, branched, cyclic, unsubstituted, substituted, and/or heteroatom-containing alkenyl and lower alkenyl groups, respectively.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” intends an alkynyl group of 2 to 6 carbon atoms. The term “substituted alkynyl” refers to an alkynyl group substituted with one or more substituent groups, and the terms “heteroatom-containing alkynyl” and “heteroalkynyl” refer to alkynyl in which at least one carbon atom within the carbon chain is replaced with a heteroatom. If not otherwise indicated, the terms “alkynyl” and “lower alkynyl” include a linear, branched, unsubstituted, substituted, and/or heteroatom-containing alkynyl and lower alkynyl group, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms as set forth above. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group, as set forth above, bound through a single, terminal ether linkage.

The term “aromatic” refers to the ring moieties which satisfy the Hückel 4n+2 rule for aromaticity, and includes both aryl (i.e., carbocyclic) and heteroaryl (also called heteroaromatic) structures, including aryl, aralkyl, alkaryl, heteroaryl, heteroaralkyl, or alk-heteroaryl moieties.

The term “aryl” as used herein, and unless otherwise specified, refers to an aromatic substituent or structure containing a single aromatic ring or multiple aromatic rings that are fused together, directly linked, or indirectly linked (such that the different aromatic rings are bound to a common group such as a methylene or ethylene moiety). Unless otherwise modified, the term “aryl” refers to carbocyclic structures. Preferred aryl groups contain 6 to 24 carbon atoms, and particularly preferred aryl groups contain 6 to 14 carbon atoms. Exemplary aryl groups contain one aromatic ring or two fused or linked aromatic rings. e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 6 to 24 carbon atoms, and particularly preferred aryloxy groups contain 6 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkylene group with an aryl substituent, wherein “aryl” and “alkyl” are as defined above. As such, alkaryls and aralkyls represent one type of substituted aryls and alkyls, respectively. Preferred alkaryl and aralkyl groups contain 7 to 25 carbon atoms, and particularly preferred alkaryl and aralkyl groups contain 7 to 17 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)— alkyl, —O(CO)-aryl, or —O(CO)-aralkyl, wherein “alkyl,” “aryl, and “aralkyl” are as defined above.

The terms “cyclic” and “ring” refer to alicyclic or aromatic groups that may or may not be substituted and/or heteroatom-containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic, or polycyclic. The term “acyclic” refers to a structure in which a double bond may or may not be contained within the ring structure.

The terms “halo.” “halide.” and “halogen” are used in the conventional sense to refer to a chloro, bromo, fluoro, or iodo substituent.

The term “heteroatom-containing” refers to a hydrocarbon molecule in which one or more carbon atoms is replaced with an atom other than carbon. e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are contain at least one heteroatom within the aromatic ring structure. It should be noted that a “heterocyclic” group or compound may or may not be aromatic, and further that “heterocycles” may be monocyclic, bicyclic, or polycyclic as described above with respect to the term “aryl.”

The term “heterocyclic” or the like refers both to heteroaromatic rings (also known as heteroaryls) and heterocycloalkyl rings (also known as heteroalicyclic groups) each heterocyclic ring containing 4 to 10 atoms in its ring system and containing one to four heteroatoms (e.g., O, N, S, and Si) in the ring(s), provided that no ring contains two adjacent O or S atoms. Non-aromatic heterocyclic groups (also known as heterocycloalkyls) include groups having only 3 atoms in their ring system, but aromatic heterocyclic groups must have at least 5 atoms in their ring system to have aromatic character described elsewhere herein. The heterocyclic groups may also include benzo- and pyridinyl-fused ring systems. Aziridinyl and azetidinyl are examples of a 3- and 4-membered heterocyclic group, respectively.

Non-limiting examples of heteroaryl substituents include optionally substituted furanyl, an optionally substituted pyridyl, an optionally substituted pyrazinyl, an optionally substituted pyrimidinyl, an optionally substituted pyridazinyl, an optionally substituted thiophenyl (thienyl), an optionally substituted pyrrolyl, an optionally substituted imidazolyl, an optionally substituted thiazolyl, an optionally substituted oxazolyl, an optionally substituted pyrazolyl, an optionally substituted isothiazolyl, an optionally substituted 1,2,3-triazolyl, an optionally substituted 1,2,4-triazolyl, an optionally substituted 1,3,4-triazolyl, an optionally substituted tetrazolyl, an optionally substituted 1,2,3-thiadiazolyl, an optionally substituted 1,2,3-oxadiazolyl, an optionally substituted 1,3,4-thiadiazolyl, an optionally substituted 1,3,4-oxadiazolyl, an optionally substituted indolyl, an optionally substituted indolinyl, an optionally substituted quinolyl, an optionally substituted tetrahydroquinolyl, an optionally substituted 1,2,3,4-tetrahydroisoquinolyl, an optionally substituted cinnolinyl, an optionally substituted quinoxalinyl, an optionally substituted quinazolinyl, an optionally substituted phthalazinyl, an optionally substituted 1,8-naphthyridinyl, an optionally substituted 1,4-benzodioxanyl, an optionally substituted coumarin, an optionally substituted dihydrocoumarin, an optionally substituted 1,5-naphthyridinyl, an optionally substituted benzofuryl, an optionally substituted 2,3-dihydrobenzofuryl, an optionally substituted 1,2-benzisoxazolyl, an optionally substituted benzothienyl, an optionally substituted benzoxazolyl, an optionally substituted benzothiazolyl, an optionally substituted purinyl, an optionally substituted benzimidazolyl, an optionally substituted benzotriazolyl, an optionally substituted thioxanthinyl, an optionally substituted carbazolyl, an optionally substituted carbolinyl, an optionally substituted acridinyl, an optionally substituted pyrrolizidinyl, or an optionally substituted quinolizidinyl.

Examples of heteroatom-containing alicyclic substituents include, but are not limited to pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxiranyl, oxetanyl, thiiranyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, and dithiolanyl.

By “substituted” as in “substituted alkyl.” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups referred to herein as “Fn,” that includes halo. C₁-C₂₄ alkoxy, C₂-C₂₄ alkenyloxy, C₆-C₂₄ aryloxy, C₇-C₂₄ aralkyloxy, C₇-C₂₄ alkaryloxy, acyl (including C₁-C₂₅ alkylcarbonyl (—CO—C₁-C₂₄ alkyl) and C₇-C₂₅ arylcarbonyl (—CO—C₆-C₂₄ aryl)), acyloxy (—O-acyl, including C₂-C₂₅ alkylcarbonyloxy (—O—CO—C₁-C₂₄ alkyl) and C₇-C₂₅ arylcarbonyloxy (—O—CO—C₆-C₂₄ aryl)), C₂-C₂₅ alkoxycarbonyl ((CO)—O—C₁-C₂₄ alkyl), C₇-C₂₅ aryloxycarbonyl (—(CO)—O—C₆-C₂₄ aryl), cyano(—C═N), cyanato (—O—C═N), thiocyanato (—S—C═N), or nitro (—NO₂).

The term “Fn” also includes the hydrocarbon moieties C₁-C₂ alkyl (preferably C₁₋₁₂ alkyl, more preferably C₁₋₆ alkyl), C₁₋₁₂ alkylene, more preferably C₁₋₆ alkylene). C₂₋₂₄ alkenyl (preferably C₂₋₁₂ alkenyl, more preferably C₂₋₆ alkenyl), C₂₋₂₄ alkenylene (preferably C₂₋₁₂ alkenylene, more preferably C₂₋₆ alkenylene), C₂₋₂₄ alkynyl (preferably C₂₋₁₂ alkynyl, more preferably C₂₋₆ alkynyl). C₆₋₂ aryl (preferably C₆₋₁₂ aryl), C₆₋₂₄ arylene (preferably C₆₋₁₂ arylene), C₇₋₂₄ alkaryl (preferably comprising a C₆₋₁₂ aryl), and C₆-C₂₄ aralkyl (preferably comprising a C₆₋₁₂ aryl). Within these substituent structures, the “alkyl.” “alkylene.” “alkenyl,” “alkenylene.” “alkynyl,” “alkynylene,” “alkoxy,” “aromatic,” “aryl,” “aryloxy.” “alkaryl,” and “aralkyl” moieties are optionally fluorinated or perfluorinated.

Within this context. “Fn” includes, but is not limited to adamantyl, chloro, fluoro, branched or cyclic C₃₋₁₂ alkyl, partially or fully fluorinated alkyl (e.g., —CF₃) C₁₋₆ alkylene, partially or fully fluorinated alkaryl (e.g., 4-phenyl-CF₃).

By “functionalized” as in “functionalized alkyl,” “functionalized olefin,” “functionalized cyclic olefin,” and the like, is meant that in the alkyl, olefin, cyclic olefin, or other moiety, at least one hydrogen atom bound to a carbon (or other) atom is replaced with one or more functional groups such as those described herein and above. The term “functional group” is meant to include any functional species that is suitable for the uses described herein.

In addition, the aforementioned functional groups may, if a particular group permits, be further substituted with one or more additional functional groups or with one or more hydrocarbyl moieties such as those specifically enumerated above. Analogously, the above-mentioned hydrocarbon moieties may be further substituted with one or more functional groups or additional hydrocarbon moieties such as those specifically enumerated.

A “neutral electron donor ligand” is any ligand which, when removed from a metal center in its closed shell electron configuration, has a neutral charge, i.e., is a Lewis base.

In independent embodiments, the neutral electron donor comprises a phosphine, sulfonated phosphine, phosphite, phosphinite, phosphonite, arsine, stibine, ether, amine, amide, imine, sulfoxide, carboxyl, carbonyl, nitrosyl, a heterocycle containing nitrogen (e.g., pyridine), sulfur (e.g., thiophene), oxygen (e.g., tetrahydrofuran), or a mixture thereof (for example, oxazoline), or thioether. In some embodiments, L is phosphine, sulfonated phosphine, phosphite, phosphinite, phosphonite, arsine, stibine, ether, (including cyclic ethers), amine, amide, imine, sulfoxide, carboxyl, nitrosyl, pyridine, substituted pyridine, imidazole, substituted imidazole, pyrazine, substituted pyrazine or thioether. Exemplary ligands are trisubstituted phosphines. Other examples are cited elsewhere herein.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes separate embodiments where a non-hydrogen substituent is present and those where a non-hydrogen substituent is not present.

In some embodiments, L is a heterocycle containing nitrogen, sulfur, oxygen, or a mixture thereof. Examples of nitrogen-containing heterocycles appropriate for L include pyridine, pyridazine, pyrimidine, pyrazine, 1,3,5-triazine, 1,2,4-triazine, 1,2,3-triazine, pyrrole, 2H-pyrrole, 3H-pyrrole, pyrazole, 2H-imidazole, 1,2,3-triazole, 1,2,4-triazole, indole, 3H-indole, 1H-isoindole, cyclopenta(b)pyridine, indazole, quinoline, bisquinoline, isoquinoline, bisisoquinoline, cinnoline, quinazoline, naphthyridine, piperidine, piperazine, pyrrolidine, pyrazolidine, quinuclidine, imidazolidine, picolylimine, purine, benzimidazole, and bisimidazole. Additionally, the nitrogen-containing heterocycles may be optionally substituted.

Examples of sulfur-containing heterocycles appropriate for L include thiophene, 1,2-dithiole, 1,3-dithiole, thiepin, benzo(b)thiophene, benzo(c)thiophene, thionaphthene, dibenzothiophene, 2H-thiopyran, 4H-thiopyran, and thioanthrene.

Examples of oxygen-containing heterocycles appropriate for L include 2H-pyran, 4H-pyran, 2-pyrone, 4-pyrone, 1,2-dioxin, 1,3-dioxin, oxepin, furan, 2H-1-benzopyran, coumarin, coumarone, chromene, chroman-4-one, isochromen-1-one, isochromen-3-one, xanthene, tetrahydrofuran, 1,4-dioxan, and dibenzofuran.

Examples of mixed heterocycles appropriate for L include isoxazole, oxazole, thiazole, isothiazole, 1,2,3-oxadiazole, 1,2,4-oxadiazole, 1,3,4-oxadiazole, 1,2,3,4-oxatriazole, 1,2,3,5-oxatriazole, 3H-1,2,3-dioxazole, 3H-1,2-oxathiole, 1,3-oxathiole, 4H-1,2-oxazine, 2H-1,3-oxazine, 1,4-oxazine, 1,2,5-oxathiazine, o-isooxazine, phenoxazine, phenothiazine, pyrano[3,4-b]pyrrole, indoxazine, benzoxazole, anthranil, and morpholine

The following Embodiments are intended to complement, rather than displace or supersede, any previous descriptions. As such, they are representative of the total disclosure, but not limiting to the specific examples set forth herein.

Embodiment 1. The present disclosure is directed to methods of preparing compounds, especially sterically hindered compounds, of the general Formula (C)

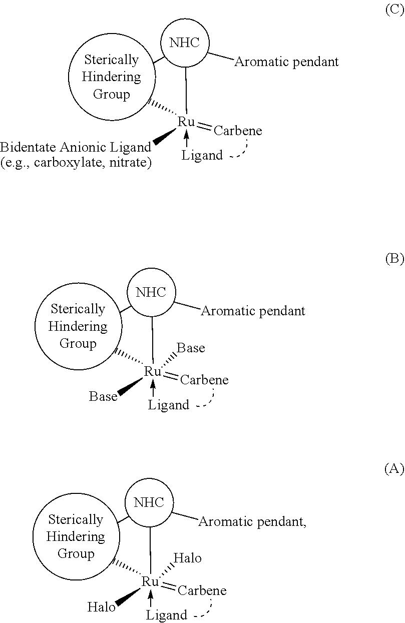

the method comprising a series of steps including the step of reacting a compound of the corresponding general Formula (B) with a carboxylic acid to form the compound of general Formula (C), where the bidentate anionic ligand is a carboxylate. The compound of the general Formula (B) may be formed by reacting a dihalo compound of the general corresponding Formula (A) with an anionic base, capable of displacing the halo ligands and coordinating to the ruthenium center as monodentate ligands to form an optionally isolated intermediate. Subsequent reactions can be used to displace the carboxylate ligand with other bidentate anionic ligands. Each process step is individually considered an independent Aspect of this disclosure. Alternatively or additionally, two or more process steps when considered together is considered a separate Aspect of this disclosure. Each of the general descriptors represented in these general Formulae (A) and (B) are set forth elsewhere herein and each and every structure arising from any combination of these descriptions are considered independent Aspects of this disclosure, both in terms of compositions and method steps.

Within this general context, certain Aspects of this Embodiment include those methods of preparing a compound of Formula (III) or (XIII), the method comprising contacting a compound of Formula (II) or (XII), respectively, or geometric isomer thereof with a carboxylic acid of formula Z—H, where Z—H is C(R¹⁸)(R¹⁹)(R²⁰)COOH, wherein the contacting results in the formation of the compound of Formula (IV) or (XIV), respectively, or geometric isomer thereof:

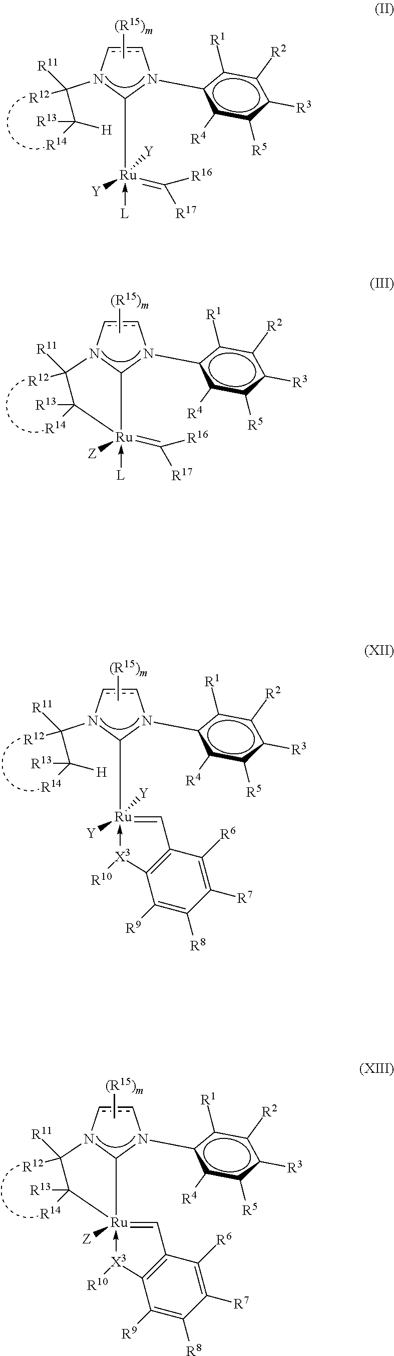

wherein:
R¹, R², R³, R⁴, R⁵, R⁶, R⁷, R⁸, and R⁹ independently are or include hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups;

and/or one or more of R¹ and R² or R² and R³ or R³ and R³ or R⁴ and R⁵ or R⁶ and R⁷ or R⁷ and R⁸ or R⁸ and R⁹, together with the carbons to which they are bound, form at least one optionally substituted 5- to 7-membered ring structure;
X³ is —O—, —S—, or —N(R^{10A})—;
R¹⁰ and R^{10A} independently are or include optionally substituted linear, branched, cyclic, or polycyclic alkyl, optionally substituted aryl, or optionally substituted heteroaryl;
R¹¹, R¹², R¹³, and R¹⁴ independently are or include hydrogen, halogen, optionally substituted alkyl, optionally substituted alkenyl, optionally substituted alkoxyl, optionally substituted aryl, optionally substituted heteroaryl; and/or
one or more pairs of R¹¹, R¹², R¹³, and R¹⁴ are independently optionally linked together to form a cyclic or polycyclic structure;
the designation [symbol] refers to the presence of an optional double bond:
m is 0, 1, 2, 3, or 4;
R¹⁵ independently is or includes an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure (e.g., comprising linked alkylenes or ethers);
Y independently is or includes a basic anion coordinated as a ligand to the ruthenium center;
R¹⁶ and R¹⁷ independently are or include hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy;
Z is a carboxylate, optionally of formula C(R¹⁸)(R¹⁹)(R²⁰)C(═O)O—;
wherein R¹⁸, R¹⁹, and R²⁰ independently are or include hydrogen, optionally substituted alkyl, optionally substituted alkenyl, optionally substituted aryl, or optionally substituted heteroaryl, provided that not all of R¹⁸, R¹⁹, and R²⁰ are hydrogen; and/or
two or three of R¹⁸, R¹⁹, and R²⁰ are linked together to form a cyclic or polycyclic structure; and
L is a donor ligand, optionally a neutral electron donor ligand, optionally a neutral electron donor solvent or an electron donor ligand linked to R¹⁷.

As presented in this Embodiment, and throughout this disclosure with respect to the other Aspects and Embodiments set forth elsewhere herein, in certain Aspects, the ruthenium compounds are presented in terms of specific NHCs (N-Heterocyclic carbenes) and aromatic pendants therefrom, though it should be appreciated that the disclosure is not necessarily limited to these structures, and also embraces those compounds, for example, where the NHC ring is a 6-membered ring optionally substituted with one or more R¹⁵ substituents.

As used throughout, the representations of the NHC core and its associated descriptors:

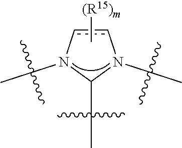

includes at least the structures where the ring independently and alternatively contains a single or a double bond. Within the context of (R¹⁵)ₘ, the difference also manifesting itself with different potential positions and distributions of the R¹⁵ groups:

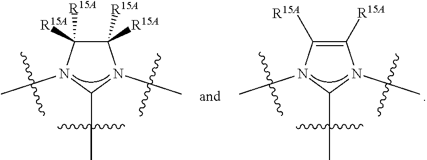

When these specific positions are represented as such, R¹⁵ is redefined as R^{15A} (if only to reflect that the absence of a non-hydrogen substituent is H) that includes the same options as for R¹⁵, but with the additional option that R^{15A} may be H, including that any two of R^{15A} may be linked to form a cyclic structure. Each of these moiety structures are considered independent of one another and their definitions may include any one or more of these structures, including all of these structures, or may be defined in terms of a group in which one or more of these independent structures may be excluded.

Non-limiting exemplars of these definitions include:

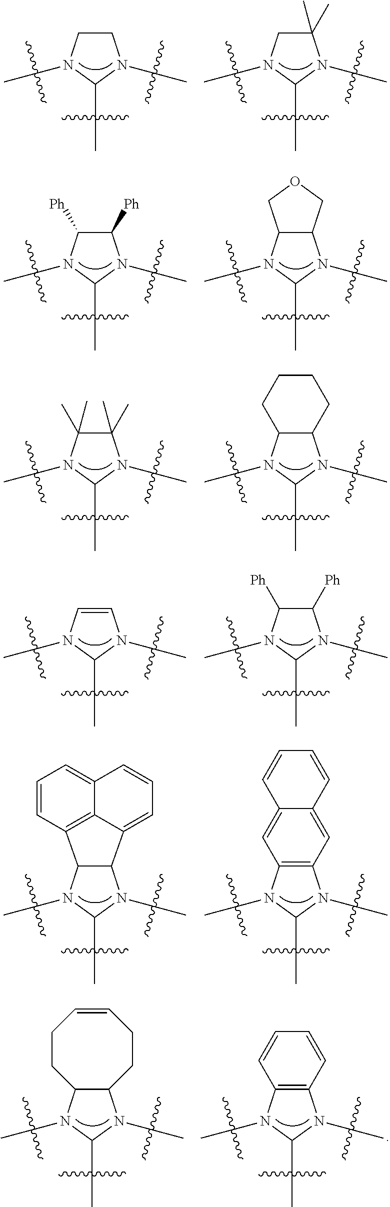

As presented in this Embodiment, and throughout this disclosure with respect to the other Aspects and Embodiments set forth elsewhere herein, in certain Aspects, R¹⁵ and R^{15A} are often described in terms of independently comprising an optionally substituted alkyl, optionally substituted alkenyl, optionally substituted alkoxy, optionally substituted aryl, optionally substituted aryloxy, optionally substituted heteroaryl, or optionally substituted heteroaryloxy, or any two of R¹⁵ and R¹“A may be linked to form a cyclic structure. It should be appreciated, though, that the definitions of R¹⁵ and R^{15A} is not necessarily limited to these functional groups and may additionally or alternatively one or more of the functional groups designated as “Fn” elsewhere herein, including for example cyano, halo, hydroxy, nitro, and trifluoromethane.

As presented in this Embodiment, and throughout this disclosure with respect to the other Aspects and Embodiments set forth elsewhere herein, in certain Aspects, the ruthenium compounds are presented in terms of optionally substituted aryl groups pendent from the NHCs, though it should be appreciated that the disclosure is not necessarily limited to these structures, and also embraces those compounds, for example, where the aromatic pendant comprises an optionally substituted heteroaryl group, as well as the aryl group depicted. Representative heteroaryl groups are set forth elsewhere in this disclosure.

Additionally or alternatively, as used herein, in the context of the Aromatic pendant, the definitions of R¹, R², R³, R⁴, and R⁵ provide that R¹ and R² or R² and R³ or R³ and R⁵ or R⁴ and R⁵, together with the carbons to which they are bound, form at least one optionally substituted 5- to 7-membered ring structure. Such optionally substituted 5- to 7-membered ring structures include, for example, optionally substituted aryl, heteroaryl, cyclic alkylene, cyclic alkenylene, or heterocyclic (e.g., fused methylene, ethylene, or propylene glycol) moieties fused to the aromatic pendent group.

Further, as presented in this Embodiment, and throughout this disclosure with respect to the other Aspects and Embodiments set forth elsewhere herein, in certain Aspects, R¹, R², R³, R⁴, R⁵, R⁶, R⁷, RH, and R⁹ are often set forth as independently hydrogen, halogen, optionally substituted alkyl (including optionally fluorinated or perfluorinated, e.g., —CF₃), optionally substituted alkenyl, optionally substituted alkoxy, optionally substituted aryl, optionally substituted aryloxy, optionally substituted heteroaryl, optionally substituted heteroaryloxy, or nitro groups. However, the definitions of these variables are not necessarily limited to these functional groups and may additionally or alternatively one or more of the functional groups designated as “Fn” elsewhere herein, including for example cyano and hydroxy.

As presented in this Embodiment, and throughout this disclosure with respect to the other Aspects and Embodiments set forth elsewhere herein, in certain Aspects, the Sterically Hindering Group pendant to the NHCs and, in some cases, bonded to the ruthenium center, is sometimes presented in terms of the structures:

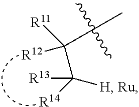

where R¹¹, R¹², R¹³, and R¹⁴ are often defined herein as independently hydrogen, halogen, optionally substituted alkyl, optionally substituted alkenyl, optionally substituted alkoxyl, optionally substituted aryl, optionally substituted heteroaryl. However, the definitions of R¹¹, R¹², R¹³, and R¹⁴ are not necessarily limited to these functional groups and may additionally or alternatively one or more of the functional groups designated as “Fn” elsewhere herein, including for example cyano, hydroxy, nitro, and trifluoromethane.

R¹¹, R¹², R¹³, and R¹⁴ are also defined as above and/or one or more pairs of R¹¹, R¹², R¹³, and R¹⁴ are independently optionally linked together to form a cyclic or polycyclic structure. The dashed line joining R¹² and R¹⁴ should not be seen as limiting, as it represents one possible linking. Again, any two or more of R¹¹, R¹², R¹³, and R¹⁴ can be linked to form cyclic or polycyclic structures. This disclosure features adamantane as one possible structure deriving from these options, though other optionally substituted bicyclic or polycyclic structures are also useful in the present methods. For example, optionally alkyl substituted bicyclopentanes, bicyclohexanes, bicycloheptanes, and bicyclooctanes are known to be used in the context of such compounds in the context of ruthenium metathesis catalyst and each are considered within the scope of the definition of this moiety. Non-limiting exemplars of within this context include:

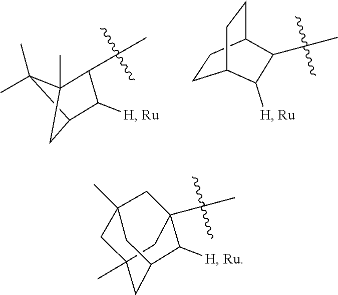

Alternatively, the definitions of R¹¹, R¹², R¹³, and R¹⁴ can also embrace aromatic structures (e.g., where R¹¹ and R¹² form part of an aromatic group) such as:

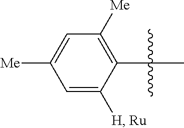

As presented in this Embodiment, and throughout this disclosure with respect to the other Aspects and Embodiments set forth elsewhere herein, in certain Aspects, the ruthenium compounds are presented in terms of optionally substituted carbene structures as:

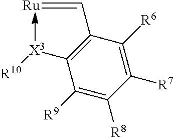

though it should be appreciated that the disclosure is not necessarily limited to these aryl structures, and also embraces those compounds, for example, where the 6-membered carbocyclic aromatic group is replaced by an optionally substituted heteroaryl group. Representative heteroaryl groups are set forth elsewhere in this disclosure. Within this context, the ruthenium carbene may comprise the structures:

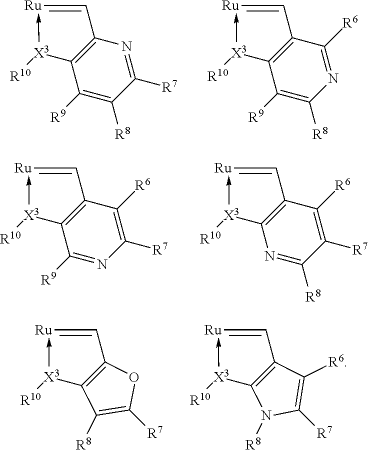

Additionally or alternatively, the definitions of R⁶, R⁷, R⁸, and R⁹ provide that R⁶ and R⁷ or R⁷ and R⁸ or R⁸ and R⁹, together with the carbons to which they are bound, form at least one optionally substituted 5- to 7-membered ring structure. That said, the carbene structures defined in terms of X³, R⁶, R⁷, R⁸, R⁹, and R¹⁰ embrace those structures in which the designated phenyl (or pyridinyl) group includes, for example, a fused optionally substituted aryl, heteroaryl, cyclic alkylene, cyclic alkenylene, or heterocyclic moiety. Exemplary non-limiting structures falling within this description include, for example optionally substituted quinolines. (di)benzofurans, (di)benzothiophenes, (di)benzopyrroles, naphthalenes, anthracenes, phenanthrenes, tetrahydronaphthalenes, dihydronaphthalenes, chromanes, benzodioxines dihydrobenzodioxines, indenes, dihydroindenes, benzodioxoles, dihydrobenzofurans, and dihydroisobenzofurans

Also as presented in this Embodiment, and throughout this disclosure with respect to the other Aspects and Embodiments set forth elsewhere herein, in certain Aspects, the ruthenium carbenes are presented in terms of both:

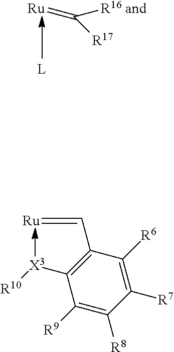

It should be appreciated that within the full context of the definitions of L, R¹⁶, and R¹⁷, the structures embracing these terms represent genera of, and embrace, the structures defined in terms of X³, R⁶, R⁷, R⁸, R⁹, and R¹⁰. To the extent that the genus is broader than the species structures, other species include those structures encompassing the difference between the two groups; e.g., those compounds included within the genus embracing the L, R¹⁶, and R¹⁷ moieties but not in the species embraced by the X³, R⁶, R⁷, R⁸, R⁹, and R¹⁰ moieties. Included within these compounds are those where the ruthenium carbene moiety include Fischer-type carbenes (in which R¹⁶ or R¹⁷ comprises an optionally substituted alkoxy, aryloxy, or heteroaryloxy pendant) or a structures in which the monodentate ligand L is absent or displaced by a heterocyclic moiety linked to the carbene group by an alkyl or ether linkage, for example:

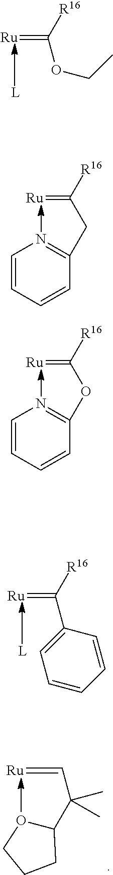

These structures are not considered limiting with respect to the disclosure, but rather represent examples of the larger scope embraced by the definitions.

Also as presented in this Embodiment, and throughout this disclosure for each of the compounds set forth herein, in various Aspects, L is defined in terms of a neutral electron donor ligand, optionally a neutral electron donor solvent or an electron donor ligand linked to R¹⁷. Also included in the context of the compounds of the present disclosure are those compounds and structures where L is absent; i.e., this group is neither provided by a carbene pendent or separately present and the ruthenium center is further coordinatively unsaturated, for example, in those cases there the steric bulk of the other moieties obstructs the inclusion of L to the ruthenium center. Where present, L may include any one or more of the neutral electron donor ligands set forth elsewhere herein. In some Aspects/Embodiments, L is an optionally substituted heteroaryl (such as pyridine, imidazole, or pyrazine; other heteroaryl options are set forth elsewhere herein), phosphine (including alkyl, aryl, and mixed alkyl/aryl phosphines), nitrile, ether (including a cyclic ether), thioether, amine, carbon monoxide, ketones (e.g., acetone), amides, sulfoxides, urea, thiourea, and N-heterocyclic carbene ligands, L may in some cases arise from the solvents used in the reactions involving these compounds, and so the definition of L includes these solvents as well.

Also as presented in these Aspects, and throughout this disclosure for each of the compounds set forth herein, X³ is defined in terms of —O—, —S—, or —N(R^{10A}). It should be understood that in each case where so provided, the definition of X³ is defined in terms of —O—, —S—, or —N(R^{10A}) individually, or as part of this group. Those Aspects/Embodiments in which X³ is —O— appear to be preferred, if only for synthetic simplicity.

Also as presented in these Aspects, and throughout this disclosure for each of the compounds set forth herein, R¹⁰ and R^{10A} are often described independently in terms of substituted alkyl, optionally substituted aryl, or optionally substituted heteroaryl. However, for the avoidance of doubt, R¹⁰ and R^{10A} may also more broadly defined to include optionally substituted C₁₋₂₄ alkyl (preferably C₁₋₁₂ alkyl, more preferably C₁₋₆ alkyl), including linear, branched, cyclic, and polycyclic alkyls, including adamantyl or other bicyclic hydrocarbons, optionally substituted C₂₋₂₄ alkenyl (preferably C₂₋₁₂ alkenyl, more preferably C₂₋₆ alkenyl), optionally substituted C₂₋₂₄ alkynyl (preferably C₂₋₁₂ alkynyl, more preferably C₂₋₆ alkynyl), optionally substituted C₆₋₂₄ aryl (preferably C₆ aryl), and optionally substituted 5-12-membered heteroaryls (preferably 5- or 6-membered heteroaryls). These structures may be optionally fluorinated or perfluorinated or otherwise substituted as set forth herein with one or more “Fn” substituents.

Further, whereas the structures set forth herein are generally represented as, for example:

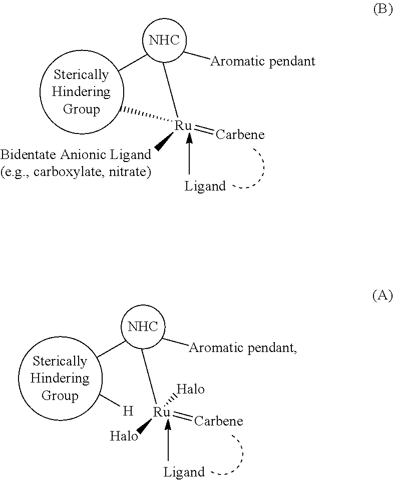

the descriptions and claims are not intended to be limited to these stereochemistries and the compounds themselves may be present as alternative geometric isomers, e.g.:

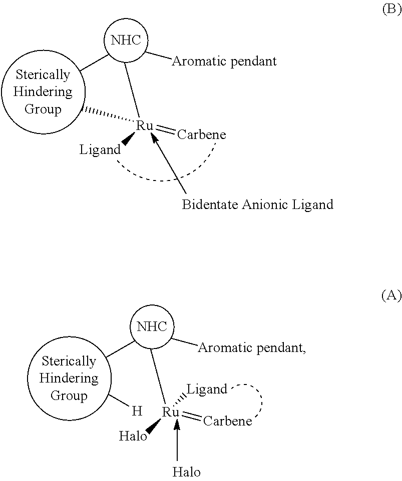

and the descriptions embrace all such geometric isomers/stereoisomers as otherwise consistent with the bonding.

As presented in these Aspects, and throughout this disclosure for each of the compounds set forth herein, particularly as relate to the compounds of Formula (II), (II-A), (XII), and (XII-A), Y is described as independently a basic anion coordinated as a ligand to the ruthenium center. In certain Aspects of this characterization, Y may be seen as relatively weakly nucleophilic but sufficiently nucleophilic to displace halide from a corresponding Ru center (as set forth elsewhere herein) and sufficiently basic to be protonated by a carboxylic acid. Alternatively or additionally, the pKa range for the conjugate acid of these bases is preferably in a range of from 10 to 19 in H₂O. For example, where Y is tert-butoxide or pyrrolate, pKa(tert-butanol)=17, and pKa(pyrrole)=17.5. This will make them sufficiently basic to react with carboxylic acid (pKa 4-5), but not too basic to decompose the Ru carbene catalyst.

Alternatively or additionally, Y may be independently a hydroxide. C₁₋₁₂ alkoxide, an amidate anion, substituted or unsubstituted phenoxide, substituted or unsubstituted pyrrolate, substituted or unsubstituted indolate, substituted or unsubstituted isoindolate, or substituted or unsubstituted imidazolate. In certain Aspects, Y is an alkoxide, an amidate anion, a phenoxide, substituted or unsubstituted pyrrolate, substituted or unsubstituted indolate, substituted or unsubstituted isoindolate, or substituted or unsubstituted imidazolate. Y may be defined in terms of any one or more of these types of anions and/or the list of options may exclude one or more type. In some Aspects the C₁₋₁₂ alkoxides include linear, branched, or cyclic alkyl alkoxides, such as methoxide, ethoxide, n-propoxide, isopropoxide, sec-butoxide, tert-butoxide, 2-pentoxide, 3-pentoxide, tert-pentoxide, tert-hexoxide, cyclopentoxide, cyclohexoxide, and (1s,4s)-bicyclo[2,2,1]heptan-1-oxide. Tert-butoxide is exemplified herein. In some Aspects, the amidate anion include bis(alkyl/silyl) amides (such as di-isopropyl amide, bis(trimethylsilyl) amide), and alkyl/aryl sulfonamide anions, such as methyl sulfonamide anions (MeSO₂NH₂ pKa=17.5) or phenyl sulfonamide anions. In some Aspects the substituted phenoxide are hindered phenoxides, including phenoxides substituted in the 2,6 positions, such as 2,6-dimethylphenoxide or 2,6-diisopropyl phenoxide)

As presented in these Aspects, and throughout this disclosure for each of the compounds set forth herein, particularly as relate to the compounds of Formula (III), (III-A), (XIII), and (XIII-A), Z is a carboxylate and Z—H is the corresponding carboxylic acid. In this disclosure, Z is generally described in terms of the formula C(R¹⁸)(R¹⁹)(R²⁰)COOH, where R¹⁸, R¹⁹, and R²⁰ are independently hydrogen, optionally substituted alkyl, optionally substituted alkenyl, optionally substituted aryl, or optionally substituted heteroaryl, provided that not all of R¹⁸, R¹⁹, and R ae hydrogen; and/or two or three of R¹⁸, R¹⁹, and R²⁰ are linked together to form a cyclic or polycyclic structure. In preferred Aspects and Embodiments, Z is a carboxylate of at least a secondary carbon group, and preferably a tertiary carbon group. Bulkier carboxylates appear to be preferred in the methods set forth herein, and pivalic acid is exemplified herein as a source of the carboxylic acid source of the pivalate. In other Aspects of these Embodiments, certain of compounds, structures, and methods set forth herein include those where Z is include isopropyl carboxylate, sec-butyl carboxylate, tert-butyl carboxylate, 2-pentyl carboxylate, 3-pentyl carboxylate, tert-pentyl carboxylate, tert-hexyl carboxylate, cyclopentyl carboxylate, cyclohexyl carboxylate. (1s,3s)-adamantane-1-carboxylate, (1s,4s)-bicyclo[2,2,1]heptane-1-carboxylate, and 1-methylcyclohexane-1-carboxylate.

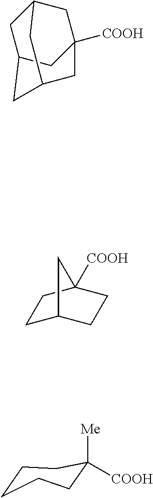 [SMILES: O=C(O)C12CC3CC(CC(C3)C1)C2 ; O=C(O)C12CCC(CC1)C2 ; CC1(C(=O)O)CCCCC1]

In some Aspects of this Embodiment, the contacting with the carboxylic is accompanied by the additional use of the corresponding carboxylate, preferably an alkali metal carboxylate.

In this Embodiment, the method is set forth as contacting a compound of Formula (II) or (XII) with a carboxylic acid of formula Z—H, wherein the contacting results in the formation of the compound of Formula (III) or (XIII), respectively. As set forth herein, the term “contacting” is intended to reflect the act of bringing the ingredients together in a manner that allows them to react with one another. Alternative language may include, reacting a compound of Formula (II) or (XII) with a carboxylic acid of formula Z—H, wherein the reaction results in the formation of the compound of Formula (III) or (XIII), respectively.” The Examples provide ample teaching to those skilled in the art of organometallic chemistry generally and ruthenium metathesis catalysts specifically to operate the methods set forth herein. Typical reactions for the present methods include reacting the ingredients at one or more temperatures in a range that may vary from −20° C., to 60° C., for a time that may vary from one or more (several) minutes to one or more (several) days. The reactions are preferably conducted under inert (e.g., argon or nitrogen) atmosphere, for example using Schlenk or glove box techniques to avoid exposure to air. Those solvents typically used in the art are useful here, including but not limited to alkanes (such as cyclohexane, hexane(s), heptane(s), pentane(s), petroleum ether), aliphatic chlorinated solvents (such as dichloromethane, dichloroethane, chloroform, carbon tetrachloride), alkyl esters (such as ethyl acetate), aliphatic nitriles (e.g., acetonitrile), amides (such as dimethylacetamide, dimethylformamide, NMP), hexamethylphosphoramide, ethers (such as diethyl ether, dimethyl ether, 1,2-dimethoxy-ethane, diethylene glycol dimethyl ether, methyl-t-butyl ether 1,4-dioxane, tetrahydrofuran, methyl-tetrahydrofuran), alcohols (e.g., C₁₋₁₂ alkanols, ethylene glycol), ketones (such as acetone), optionally substituted aromatic hydrocarbons (such as benzene, toluene, fluorobenzene, chlorobenzene, xylenes, pentafluorobenzene, hexafluorobenzene), heteroaromatic solvents (such as pyridine), sulfoxides (e.g., dimethyl sulfoxide), nitromethane, or mixtures thereof.

Embodiment 2. The method of Embodiment 1, further comprising contacting a compound of Formula (I) or (XI), or geometric isomer thereof, with a salt of formula [M⁺][Y⁻], where the anion Y⁻ corresponds to the Y ligand as set forth at least in Embodiment 1, wherein the contacting results in the formation of the compound of Formula (II) or (XII), respectively, or geometric isomer thereof:

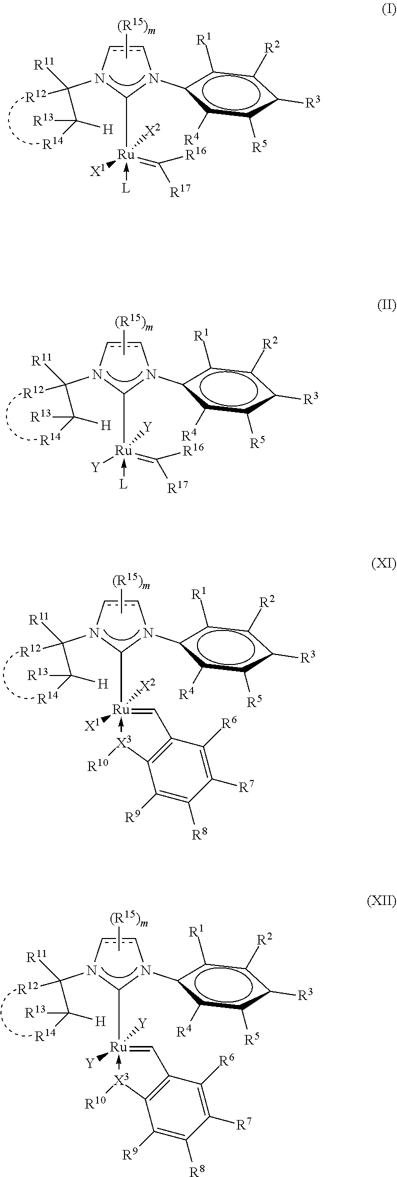

wherein:
X¹ and X² are independently chloro, bromo, or iodo;
M⁺ is monocation equivalent cation, such as an alkali metal cation, an ammonium cation, or other nitrogen-based cation, or may optionally be or comprise other cations set forth herein (e.g., di- and tri-valent cations).

In other Aspects of this Embodiment, the method may be conducted independently; i.e., not in tandem with Embodiment 1.

In certain Aspects of this Embodiment, and in the compounds and methods set forth elsewhere herein chloro is preferred for both X¹ and X² if only because of the simplicity and cost effectiveness of the making the dichloro compounds.

Also in certain Aspects of this Embodiment, the Y⁻ anion corresponds to the Y ligand as set forth in Embodiment 1, as are the compounds of Formulae (II), (II-A), (XII), and (XII-A), and those disclosures are incorporated here. Likewise, the descriptions associated with the general structures of the NHCs, the aromatic pendants, the sterically hindering group, the carbene and the ligand as set forth in Embodiment 1 for these structures also apply to the corresponding compounds of Formulae (I), (I-A), (XI), and (XI-A). Also, the reaction conditions set forth in Embodiment 1 are also applicable here, with respect to time, temperature, solvent, and protection for oxidation.

In Aspects of this Embodiment. M⁺ is defined in terms of monocation equivalent cation such as an alkali metal cation, an ammonium cation, or other nitrogen-based cation. Within this context, in certain of these Aspects. Li⁺, Na⁺, K⁺, Rb⁺, Cs⁺, optionally alkylated/arylated ammonium, or optionally substituted pyridinium cations are useful. The specific choice of a particular M⁺ cation with a given Y⁻ anion depends more on the convenience of source than the specific nature of the matched pair. Li⁺, Na⁺, K⁺ are preferred cations. In some Aspects here, [M⁺][Y⁻] include the use of potassium tert-butoxide and lithium pyrrolate. In other Aspects, the reactions set forth in this Embodiment may be conducted in the absence or presence of a cation sequestering agent, such as a cryptand or crown ether.

In some Aspects of this Embodiment, the definitions of M⁺ monocation equivalent cation are not necessarily limited to those presented in this Embodiment and throughout (i.e., alkali metal cation, ammonium cations, or other nitrogen-based cations) and other cations may be used in their stead. In other Aspects, the M⁺ monocation equivalent cation can also comprise other metallic cations, for example Ag⁺, Cu⁺, Cu²⁺, Zn²⁺, Mg²⁺, and Al³⁺, with or without other associated ligands. The term “monocation equivalent cation” refers to the presence of sufficient charge to counter the single anion Y⁻, for example ½Zn²⁺, as in ½ (Zn²⁺)(Y⁻)₂.

Embodiment 3. The method of Embodiment 1 or 2, further comprising reacting the compound of formula (III) or (XIII), or geometric isomer thereof, with a trifluoroacetate, nitrate, nitrite, oxalate, phosphate, sulfite, or sulfonate or other anion of a strong acid (H-Q), capable of coordinating the ruthenium as a bidentate ligand, to form a compound of formula (IV) or (XIV), respectively, or geometric isomer thereof:

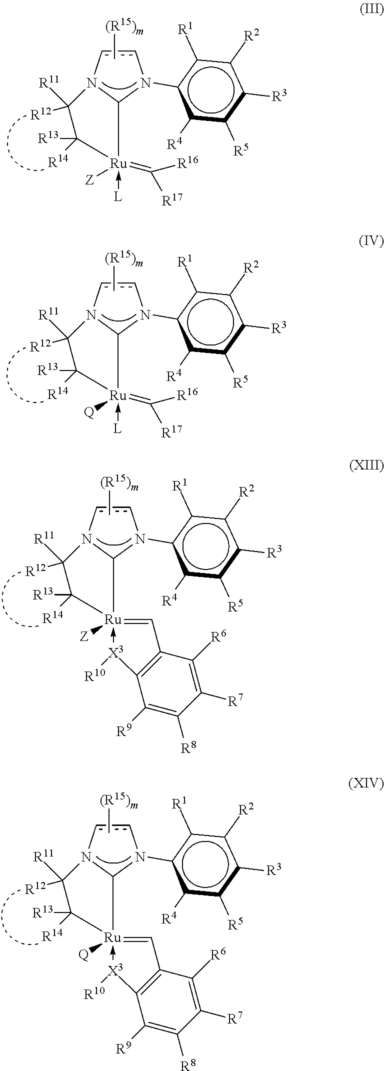

wherein Q is a coordinated trifluoroacetate, nitrate, nitrite, oxalate, phosphate, sulfite, sulfonate, or other anion of a strong acid (H-Q), capable of coordinating the ruthenium as a bidentate ligand. Typically these anions are coordinated to the ruthenium as a bidentate ligand. Nitrate appears to be preferred based on the reactivities of such complexes. Ammonium salts of these anions appear to be suitable sorts of these anions. In particular, ammonium nitrate works well in this context.

In other Aspects of this Embodiment, the method may be conducted independently; i.e., not in tandem with Embodiment 1 and/or 2.

In this Embodiment, as in the prior Embodiments, the descriptions associated with the general structures of the NHCs, the aromatic pendants, the sterically hindering group, the carbene and the ligand as set forth in Embodiment 1 also apply to the corresponding compounds of Formulae (III), (III-A), (XIII), and (XIII-A). Also, the reaction conditions set forth in Embodiment 1 are also applicable here, with respect to time, temperature, solvent, and protection for oxidation.

Embodiment 4. The method of Embodiment 2 or 3, wherein the step of contacting the compound of Formula (I) or (XI) with the salt of formula [M⁺][Y⁻], thereby resulting in the formation of the compound of Formula (II) or (XII), respectively, and the subsequent step of contacting the compound of Formula (II) or (XII), respectively, with a carboxylic acid of formula Z—H, thereby resulting in the formation of the compound of Formula (III) or (XIII) are conducted without the isolation of the compound of Formula (II) or (XII), respectively.

Embodiment 5. The method of any one of Embodiments 1 to 4, wherein R¹ and R⁴ are optionally substituted C₁₋₆ alkyl, preferably branched C₃₋₆ alkyl, branched C₄₋₆ alkyl, or phenyl, and R², R³, and R⁵ are independently hydrogen or optionally substituted C₁₋₆ alkyl, preferably branched C₃₋₆ alkyl. In other Aspects, one or more of the definitions of definitions of R¹, R², R³, R⁴ and R⁵ are excluded from the general definitions of these terms.

Embodiment 6. The method of any one of Embodiments 1 to 4, wherein R¹, R⁴, and optionally R³ are independently C₁₋₆ alkyl, preferably branched C₃₋₆ alkyl, branched C₄₋₆ alkyl, or phenyl. In certain Aspects of this Embodiment, R² and R⁵ are H. In other Aspects, one or more of the definitions of definitions of R¹, R², R³, R⁴ and R⁵ are excluded from the general definitions of these terms.

Embodiment 7. The method of any one of Embodiments 1 to 4, wherein the Aromatic pendent:

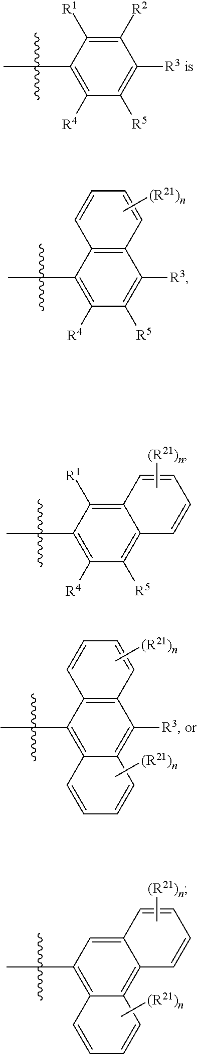

wherein R²¹ is halogen, optionally substituted C₁₋₁₂ alkyl, optionally substituted C₂₋₁₂ alkenyl, optionally substituted C₁₋₁₂ alkoxy, optionally substituted C₆₋₂₄-aryl, optionally substituted C₃₋₂₄-heteroaryl, or nitro; and
   n is independently 0 to 4 at each occurrence.

Embodiment 8. The method of any one of Embodiments 1 to 4, wherein the Aromatic pendent:

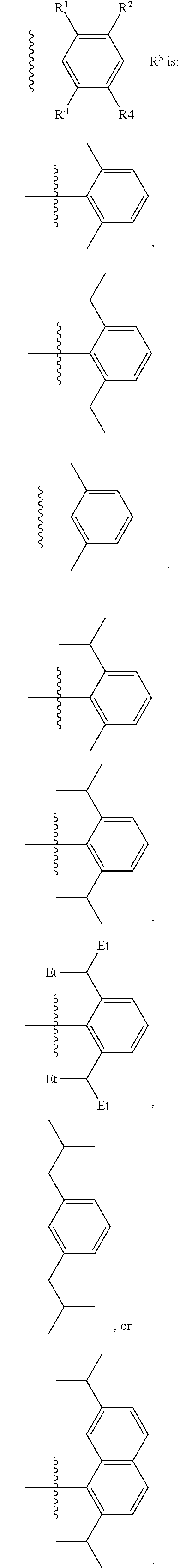

Embodiment 9. The method of any one of Embodiments 1 to 8, wherein the X³ is —O—.

Embodiment 10. The method of any one of Embodiments 1 to 9, wherein R¹¹ and/or R^{10A} is substituted or unsubstituted phenyl or a branched C₃₋₂₄ alkyl, cyclic C₃₋₂₄ alkyl, bicyclic C₆₋₂₄ alkyl, or polycyclic C₆₋₂₄ alkyl, for example isopropyl, tert-butyl, or adamantyl. In other Aspects of this Embodiment, substituted or unsubstituted phenyl or a branched C₃₋₆ alkyl, including isopropyl is excluded from the definition of R¹⁰ and/or R^{10A}.

Embodiment 11. The method of any one of Embodiments 1 to 10, wherein the Sterically Hindering Group:

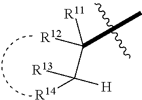

comprises an optionally alkyl substituted adamantyl moiety.

Embodiment 12. The method of any one of Embodiments 1 to 11, wherein:
(a) the compound of Formula (II) is the compound of Formula (II-A) and the compound of Formula (III) is the compound of Formula (III-A):

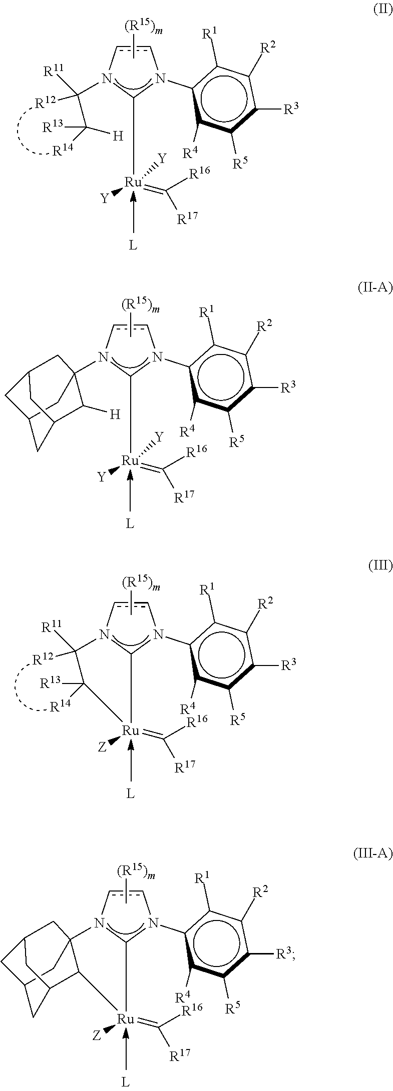

(b) the compound of Formula (XII) is the compound of Formula (XII-A) and the compound of Formula (XIII) is the compound of Formula (XIII-A):

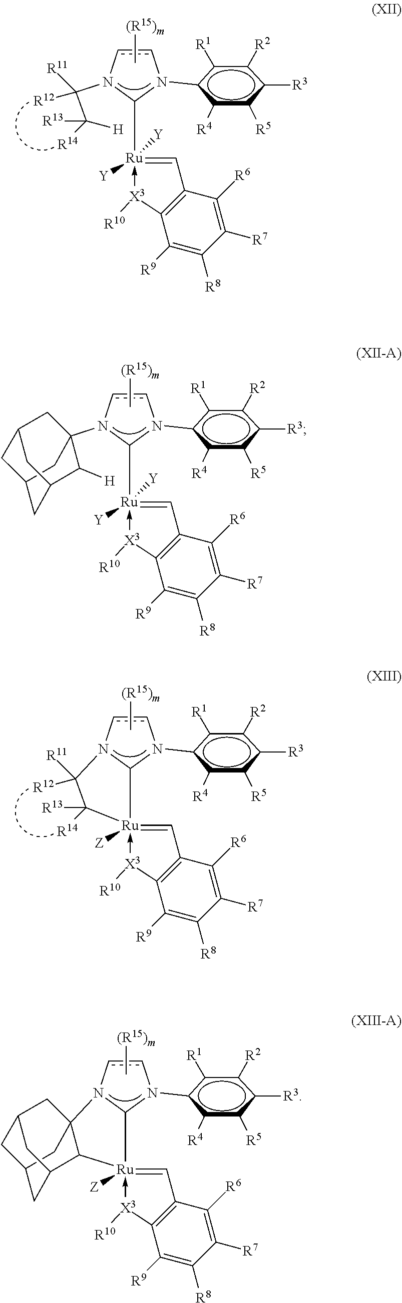

In some Aspects of this Embodiment, the Aromatic moiety is sterically bulkier than Dipp (2,6-diisopropylphenyl, i.e., R¹=R⁴=isopropyl) either by the presence of larger substituents in the R¹ and R⁴ positions (e.g., branched alkyl with more than 4 carbon; aryl or heteroaryl) or the presence of the fused C₅0.7 ring systems.

Embodiment 13. The method of any one of Embodiments 2 to 12, wherein:
(a) the compound of Formula (I) is the compound of Formula (I-A):

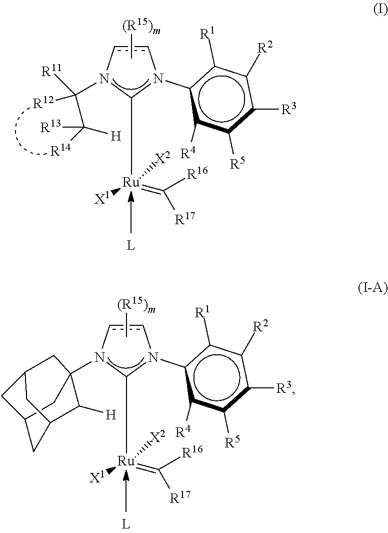

(b) the compound of Formula (XI) is the compound of Formula (XI-A):

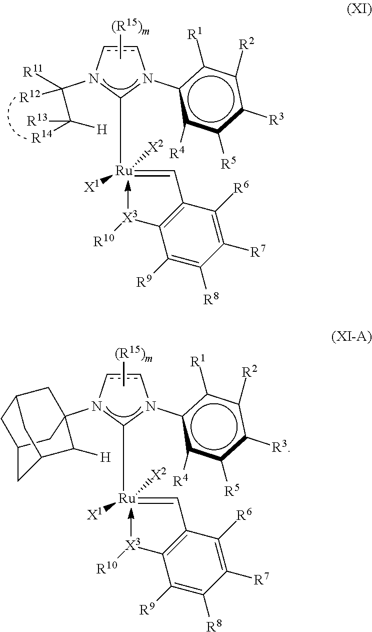

In some Aspects of this Embodiment, the Aromatic moiety is sterically bulkier than Dipp (2,6-diisopropylphenyl, i.e., R¹=R⁴=isopropyl) either by the presence of larger substituents in the R¹ and R⁴ positions (e.g., branched alkyl with more than 4 carbon; aryl or heteroaryl) or the presence of the fused C₅₋₇ ring systems.

Embodiment 14. The method of any one of Embodiments 3 to 13, wherein:
(a) the compound of Formula (IV) is the compound of Formula (IV-A):

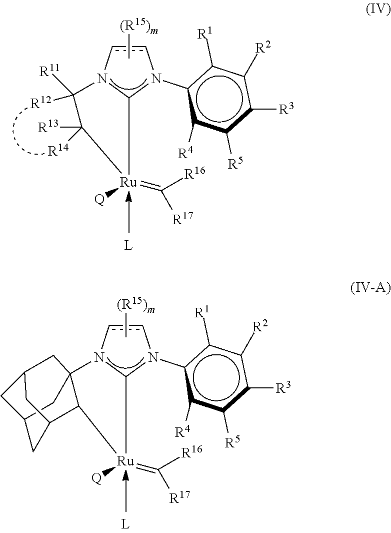

(b) the compound of Formula (XIV) is the compound of Formula (XIV-A):

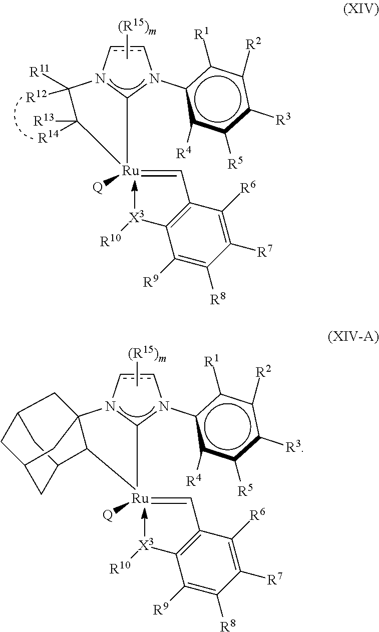

In some Aspects of this Embodiment, the Aromatic moiety is sterically bulkier than Dipp (2,6-diisopropylphenyl, i.e., R¹=R⁴=isopropyl) either by the presence of larger substituents in the R¹ and R⁴ positions (e.g., branched alkyl with more than 4 carbon; aryl or heteroaryl) or the presence of the fused C₅₋₇ ring systems.

Embodiment 15. A ruthenium compound of Formula (XX-A), (XX-B), (XX-C) or (XX-D):

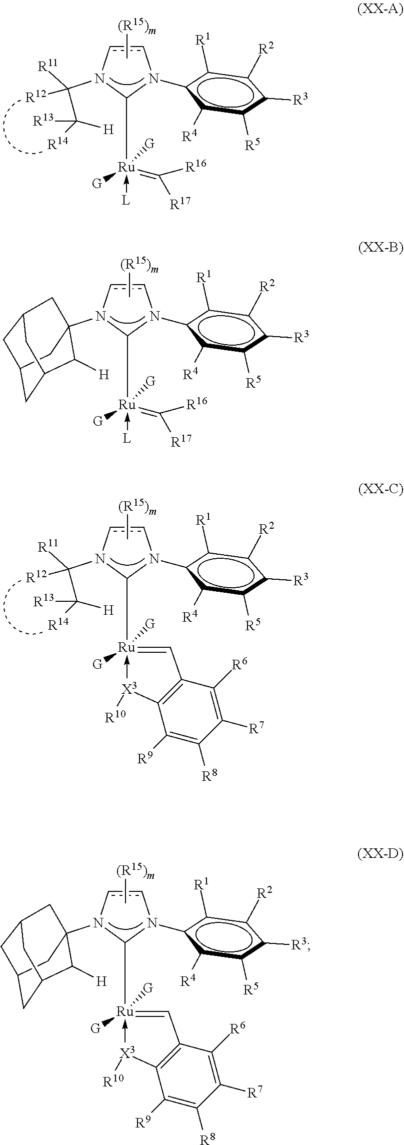

wherein:
   R¹, R², R³, R⁴, R⁶, R⁷, R⁸, and R⁹ are independently hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups;
   and/or one or more of R¹ and R² or R² and R³ or R⁶ and R⁷ or R⁷ and R⁸ or R⁸ and R⁹, together with the carbons to which they are bound, form at least one optionally substituted 5- to 7-membered ring structure;
   X³ is —O—, —S—, or —N(R^{10A})—,
   R¹⁰ and R^{10A} are optionally substituted linear, branched, cyclic, bicyclic, or polycyclic C₃₋₂₄alkyl, optionally substituted aryl, or optionally substituted heteroaryl;
   R¹¹, R¹², R¹³, and R¹⁴ are independently hydrogen, halogen, optionally substituted alkyl, optionally substituted alkenyl, optionally substituted alkoxyl, optionally substituted aryl, optionally substituted heteroaryl; and/or
   one or more pairs of R¹¹, R¹², R¹³, and R¹⁴ are independently optionally linked together to form a cyclic or polycyclic structure;
   the designation [symbol] refers to the presence of an optional double bond;
   m is 0, 1, 2, 3, or 4;
   R¹ is independently an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure (e.g., comprising linked alkylenes or ethers);
   R¹⁶ and R¹⁷ are independently hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy; G is independently:
   (a) chloro, bromo, or iodo, or a combination thereof;
   (b) a basic anion coordinated as a ligand to the ruthenium center, each basic anion comprising hydroxide, C₁₋₁₂ alkoxide, amidate anion, substituted or unsubstituted phenoxide, substituted or unsubstituted pyrrolate, substituted or unsubstituted indolate, substituted or unsubstituted isoindolate, or substituted or unsubstituted imidazolate; and
   L is a neutral electron donor ligand, optionally a neutral electron donor solvent or an electron donor ligand linked to R¹⁷.

In some Aspects of this Embodiment, the following provisos apply:
when G is a chloro, bromo, or iodo, or a combination thereof, then R¹ and R⁴ are independently optionally substituted C₄₋₁₂, C₅₋₁₂, C₆₋₁₂, C₇₋₁₂, or C₈₋₁₂ alkyl, optionally substituted aryl, optionally substituted heteroaryl, or optionally substituted heteroaryloxy; and/or one or more of R¹ and R² or R² and R³ or R³ and R⁵ or R⁴ and R⁵, together with the carbons to which they are bound, form at least one optionally substituted 5- to 7-membered ring structure.

In other Aspects of this Embodiment, when G is a chloro, bromo, or iodo, or a combination thereof, then the R¹ and R⁴ are independently optionally substituted branched C₄₋₁₂, C₅₋₁₂, C₆₋₁₂, C₇₋₁₂, or C₈₋₁₂ alkyl.

In other Aspects of this Embodiment, when G is a chloro, bromo, or iodo, or a combination thereof, then neither R¹⁰ nor R^{10A} are isopropyl or optionally substituted phenyl.

The definitions set forth in this Embodiment for R¹, R², R³, R⁴, R⁵, R⁶, R⁷, R⁸, R⁹, R¹⁰, R^{10A}, R¹¹, R², R¹³, R¹⁴, R¹⁵, R¹⁶, R¹⁷, R¹⁸, R¹⁹, R²⁰, G, L, and m are not necessarily intended to limit these compositions. For example, the definitions of G here includes those Aspects, definitions, and descriptions associated with X¹, X², and Y in the preceding Embodiments. More generally, in certain Aspects of this Embodiment, the descriptions associated with the general structures of the NHCs, the aromatic pendants, the sterically hindering group, the carbene and the ligand as set forth in any of the preceding Embodiments also apply to the corresponding compounds of Formulae (XX-A), (XX-B), (XX-C), and (XX-D), including the specific exclusions presented for certain of the definitions of R¹, R², R³, R⁴, R⁵, R⁶, R⁷, R⁸, R⁹, R¹⁰, R^{10A}, R¹¹, R¹², R¹³, R¹⁴, R¹⁵, R¹⁶, R¹⁷, R¹⁸, R¹⁹, R²⁰, G, L, and m.

Embodiment 16. A ruthenium compound of Formula (Y-A), (Y-B), (Y-C), or (Y-D):

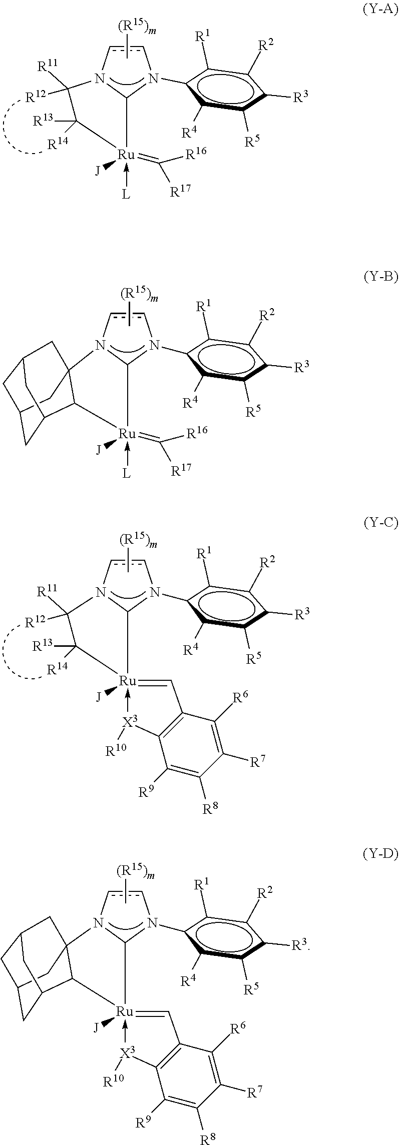

wherein:
   R¹, R², R³, R⁴, R⁵, R⁶, R⁷, R⁸, and R⁹ are independently hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups;
   and/or one or more of R¹ and R² or R² and R³ or R⁶ and R⁷ or R⁷ and R⁸ or R⁸ and R⁹, together with the carbons to which they are bound, form at least one optionally substituted 5- to 7-membered ring structure;
   X³ is —O—, —S—, or —N(R_{10A})—;
   R¹⁰ and R^{10A} are optionally substituted linear, branched, cyclic, bicyclic, or polycyclic C₃₋₂₄alkyl, optionally substituted aryl, or optionally substituted heteroaryl;
   R¹¹, R¹², R¹³, and R¹⁴ are independently hydrogen, halogen, optionally substituted alkyl, optionally substituted alkenyl, optionally substituted alkoxyl, optionally substituted aryl, optionally substituted heteroaryl; and/or
   one or more pairs of R¹¹, R¹², R¹³, and R¹⁴ are independently optionally linked together to form a cyclic or polycyclic structure;
   the designation [symbol] refers to the presence of an optional double bond;
   m is 0, 1, 2, 3, or 4;
   R¹⁵ is independently an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure (e.g., comprising linked alkylenes or ethers);
   J is independently:
   (a) a monodentate ligand, such as those set forth elsewhere herein for XI or Y, for example a phenoxide substituted at least in its 2,6-positions;
   (b) a carboxylate [optionally of formula C(R¹⁸)(R¹⁹)(R²⁰)C(═O)O—], wherein R¹⁸, R⁹, and R² are independently hydrogen, optionally substituted alkyl, optionally substituted alkenyl, optionally substituted aryl, or optionally substituted heteroaryl, provided that not all of R¹⁸, R¹⁹, and R are hydrogen; and/or two or three of R¹⁸, R¹⁹, and R²⁰ are linked together to form a cyclic or polycyclic structure; or
   (c) a trifluoroacetate, nitrate, nitrite, oxalate, phosphate, sulfite, sulfonate, or other anion of a strong acid (H-Q), capable of coordinating the ruthenium as a bidentate ligand;
   R¹⁶ and R¹⁷ are independently hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy; and
   L is a neutral electron donor ligand, optionally a neutral electron donor solvent or an electron donor ligand linked to R¹⁷.

In some Aspects of this Embodiment, the following provisos apply: when J is the substituted phenoxide, the carboxylate of formula C(R¹⁸)(R¹⁹)(R²⁰)C(═O)O—, or the trifluoroacetate, the nitrate, the nitrite, the oxalate, the phosphate, the sulfite, the sulfonate, or the other anion of a strong acid (H-Q), capable of coordinating the ruthenium as a bidentate ligand, then the Aromatic pendant is bulkier than Dipp (2,6-diisopropylphenyl), for example, then R¹ and R⁴ are independently optionally substituted C₄₋₁₂ alkyl, optionally substituted aryl, optionally substituted heteroaryl, or optionally substituted heteroaryloxy; and/or one or more of R¹ and R² or R² and R³ or R³ and R⁵ or R⁴ and R⁵, together with the carbons to which they are bound, form at least one optionally substituted 5- to 7-membered ring structure.

In some Aspects of this Embodiment, the following provisos apply: when J is the substituted phenoxide, the carboxylate of formula C(R¹⁸)(R¹⁹)(R²⁰)C(═O)O, or the trifluoroacetate, the nitrate, the nitrite, the oxalate, the phosphate, the sulfite, the sulfonate, or the other anion of a strong acid (H-Q), capable of coordinating the ruthenium as a bidentate ligand, then R¹⁰ is bulkier than isopropyl, for example optionally substituted C₄₋₁₂ alkyl, optionally substituted aryl, optionally substituted heteroaryl, or optionally substituted heteroaryloxy.

The definitions set forth in this Embodiment for R¹, R², R³, R⁴, R⁵, R¹, R⁷, R⁸, R⁹, R¹⁰, R^{10A}, R¹¹, R¹², R¹³, R¹⁴, R¹⁵, R¹⁶, R¹⁷, R¹⁸, R¹⁹, R²⁰, L, and m are not necessarily intended to limit these compositions. For example, the definitions of J here also includes those Aspects, definitions, and descriptions associated with X¹, Y, Q, and Z in the preceding Embodiments. More generally, in certain Aspects of this Embodiment, the descriptions associated with the general structures of the NHCs, the aromatic pendants, the sterically hindering group, the carbene and the ligand as set forth in any of the preceding Embodiments also apply to the corresponding compounds of Formulae (Y-A), (Y-B), (Y-C), and (Y-D), including the specific exclusions presented for certain of the definitions of R¹, R², R³, R⁴, R⁵, R⁶, R⁷, R⁸, R⁹, R¹⁰, R^{10A}, R¹¹, R¹², R¹³, R¹⁴, R¹⁵, R¹⁶, R¹⁷, R¹⁸, R¹⁹, R²⁰, Q, and Z.

Some exemplary aspects of this embodiment, based on the descriptions herein include:

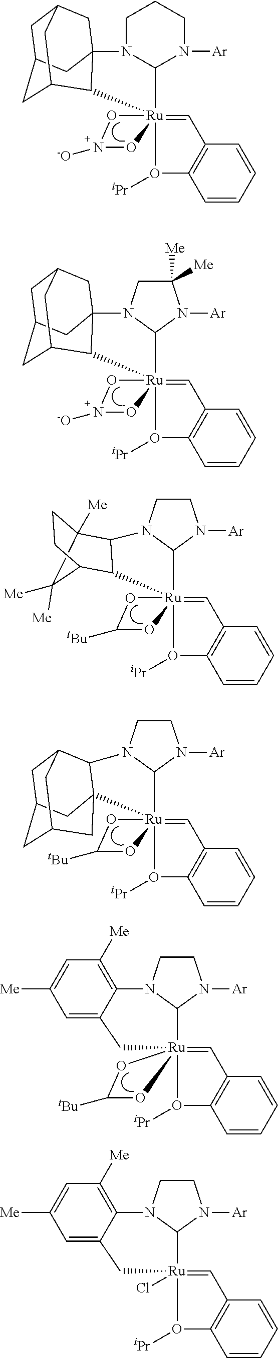

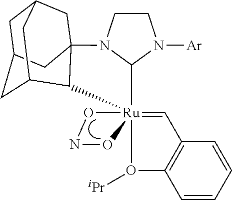

here Ar is an Aromatic pendant bulkier than Dipp or where R¹⁰ is an optionally substituted aromatic group.

Embodiment 17. A process comprising contacting at least one alkene, alkyne, or enyne with the ruthenium compound of Embodiment 15 or 16, or a ruthenium compound prepared by a method of any one of the Embodiments of 1 to 14, the contacting resulting in a reaction comprising:
(a) a cross metathesis (CM) of two different alkenes, or the alkene, alkyne, and/or enyne,
(b) a ring closing metathesis of a diene (RCM) leading to the formation of cycloalkene ring;
(c) a homodimerization of two olefins
(d) an acyclic diene metathesis (ADMET) leading to the formation of an oligomer and/or a polymer;
(e) a ring opening cross metathesis (ROCM) leading to an acyclic diene;
(f) a ring opening metathesis polymerization (ROMP) of a cycloalkene leading to a polymer; or
(g) ethenolysis of an E/Z isomer mixture of olefins to provide an enrichment of the E-olefin content of the mixture.

Each reaction process, when taken independent represents with respect to the ruthenium compounds set forth in this Embodiment, is an independent Aspect of this Embodiment as set forth elsewhere herein, as if independently enumerated.

In certain Aspects of this Embodiment, especially as related to the reactions set forth in (a) to (f), the product of the corresponding reaction is predominantly a Z-isomer olefin. For example, where the product of the reaction is or comprises a mixture of E- and Z-isomers, the Z-isomer content is greater than 50 mol % of the E-/Z- isomer mixture. In independent Aspects of this Embodiment, the Z-isomer content is 55 mol % or more, 60 mol % or more, 65 mol % or more, 70 mol % or more, 75 mol % or more, 80 mol % or more, 85 mol % or more, 90 mol % or more, 95 mol % or more, 97 mol % or more, 98 mol % or more, 99 mol % or more, or practically 100 mol % of the E-/Z-isomer mixture.

As related to the ethenolysis set forth in (g), the reaction associated with the process provides for the preferential reaction and removal of the Z-olefin from the mixture. In certain independent Aspects of this Embodiment, the contacting results in the preferential removal of the Z-olefin, such that the final proportion of the E-olefin in the remaining E-/Z-olefin mixture is at least 95%, 96%, 97%, 98%, 99%, 99.5%, 99.8%, or practically pure E-olefin (i.e., the Z-olefin has been entirely removed from the mixture)

While the catalysts described herein are considered unique for their ability to effect transformations providing Z-isomer products, metathesis reactions and the conditions for conducting them are well known for these Grubbs type catalysts. The skilled artisan would be able to conduct these reactions and recognize the advantages of these ruthenium compounds in use in the methods without undue experimentation. See, e.g., Montgomery, et al., “Recent Advancements in Stereoselective Olefin Metathesis Using Ruthenium Catalysts.” Catalysts. 2017, 7(3), 87.

### EXAMPLES

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C. pressure is at or near atmospheric.

### Example 1: General Information

All reactions were carried out in dry glassware under an inert atmosphere (e.g., nitrogen or argon) using standard Schlenk line techniques or in a Glovebox under a nitrogen atmosphere unless specified otherwise. Chemicals were received from commercial sources and purified as is standard practice in the art. Standard NMR spectroscopy experiments were conducted on a Varian Mercury (¹H, 300 MHz) spectrometer, a Varian Inova 400 MHz spectrometer, a Varian 500 MHz spectrometer equipped with an AutoX probe, or a Varian 600 MHz spectrometer equipped with a Triax Probe. Chemical shifts are reported in ppm downfield from Me₄Si by using the residual solvent peak as an internal standard. Spectra were analyzed and processed using MestReNova.

The following reactions (as represented in the various embodiments set forth in the descriptions and Figures) were carried out under inert atmosphere (nitrogen or argon) at temperatures varying from −20° C., to 40° C. The reaction times varies from several minutes to several hours, depending in part on the nature of the compositions or in some cases merely for convenience. Several representative examples described as follows, though these procedures can be modified as would be appreciated by those skilled in the art, and so should be considered exemplary rather than limiting

Example 2: Preparation of Z-olefin metathesis catalyst Ru-5: Ru-5, which itself is a Z-selective olefin metathesis catalyst and also a precursor for Ru-1 was prepared according to the following Scheme:

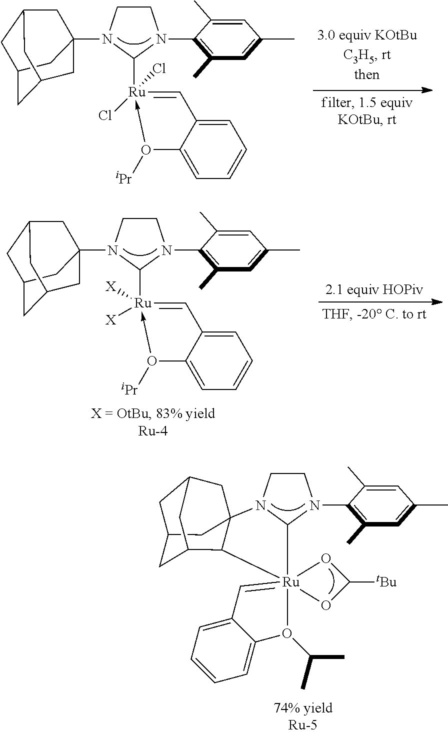

In the first reaction, the anion ligand exchange reached full conversion by treating the ruthenium dichloride with two batches of potassium tert-butoxide, furnishing the ruthenium bis-tert-butyl oxide (Ru-4) in 83% yield. In the second reaction, treating Ru-4 with pivalic acid at −20° C. led to the clean formation of a transient ruthenium bis-pivalate species, which underwent C—H activation smoothly upon warmed to room temperature, furnishing Ru-5 in 74% yield with >98% purity.

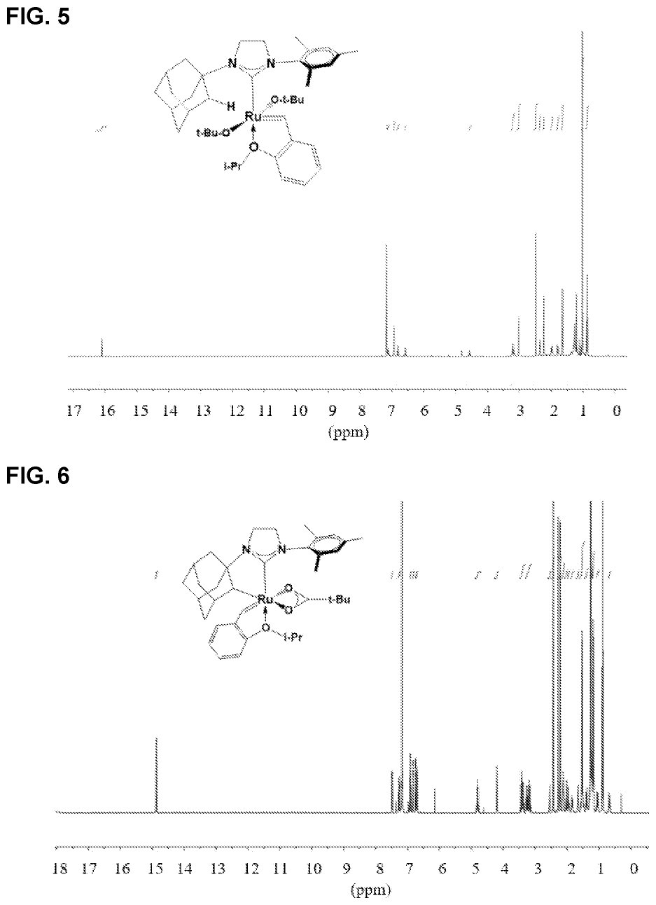

More specifically. KO-tert-Bu (1.5 mmol, 3 equiv, 168 mg) was added in one portion to a solution of starting ruthenium dichloride (0.5 mmol, 1.0 equiv, 322 mg) in benzene (8 mL) in a 20 mL vial. The suspension was stirred for 5 hours at room temperature, before filtered through a short pad of Celite into another 20 mL vial that was charged with KO-tert-Bu (0.75 mmol, 1.5 equiv, 84 mg). The resulting suspension was further stirred overnight and then concentrated in vacuo. The residue was dissolved in pentane (8 mL), passed through a short pad of Celite, and concentrated in vacuo to yield Ru-4 (ca. 90-95% purity) as a brown solid (30) mg, 83% yield). A representative ¹H-NMR spectrum of Ru-4 is provided in FIG. 5.

Next, an 8 ml vial was charged with a portion of the Ru-4 (72 mg, 0.1 mmol, 1.0 equiv) in THF (2 mL, solution A). A 4 mL vial was charged with PivOH (21.4 mg, 0.21 mmol, 2.1 equiv) in THF (1.5 mL, solution B). Both vials were put in the glovebox freezer (−20° C.) for 15 minutes. Solution B was quickly transferred into solution A by using a 3 mL syringe, and the resulting mixture was put back into the freezer and sat without stirring for 1.5 hours. The mixture was then taken out and stirred for 15 minutes at room temperature before concentrated in vacuo. The residue was transfer onto a short Celite column and flushed with pentane (3.5 mL) to remove the brownish color band and then benzene (7 mL) to obtain the product as a purple solution. Removal of the solvent furnished Ru-5 (>97% purity) as a purple solid (50 mg, 74% yield). A representative ¹H-NMR spectrum of Ru-5 is provided in FIG. 6.

Example 3: The procedure set forth in Example 2 was also applied to the synthesis of a new type of Z-selective olefin metathesis catalysts bearing fast-initiating ligands,

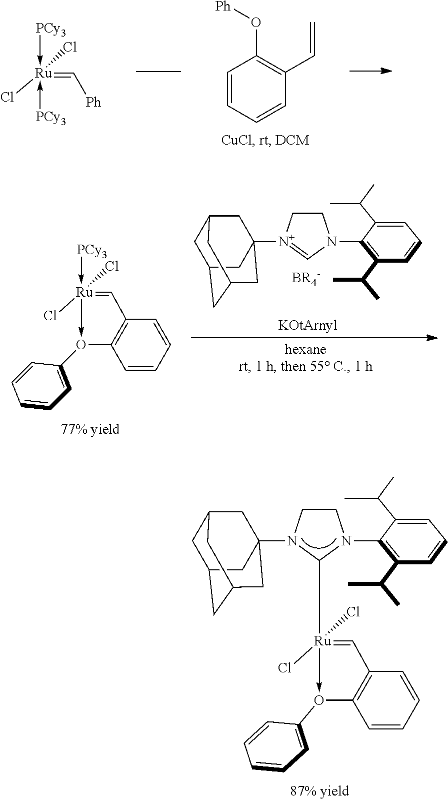

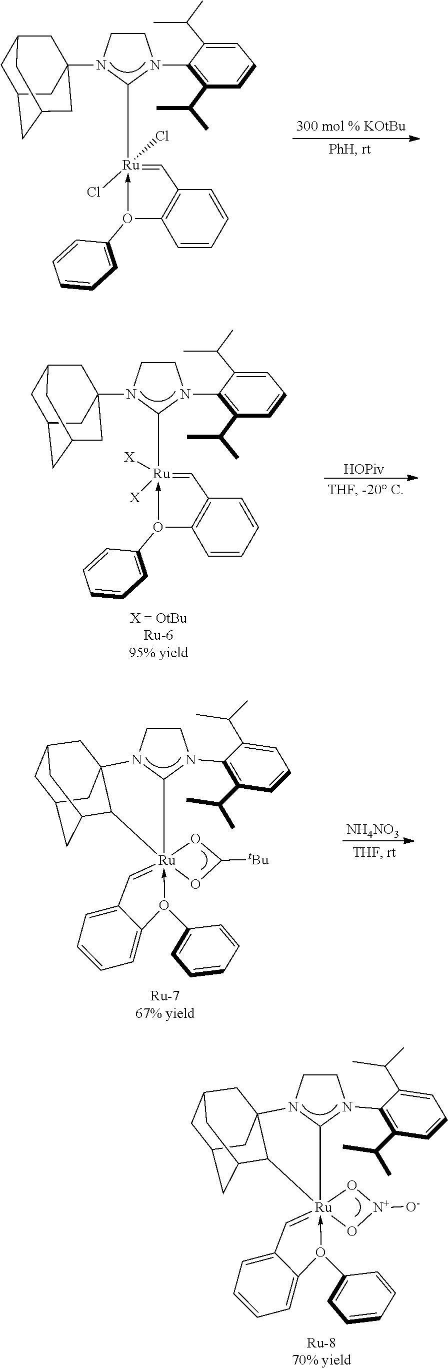

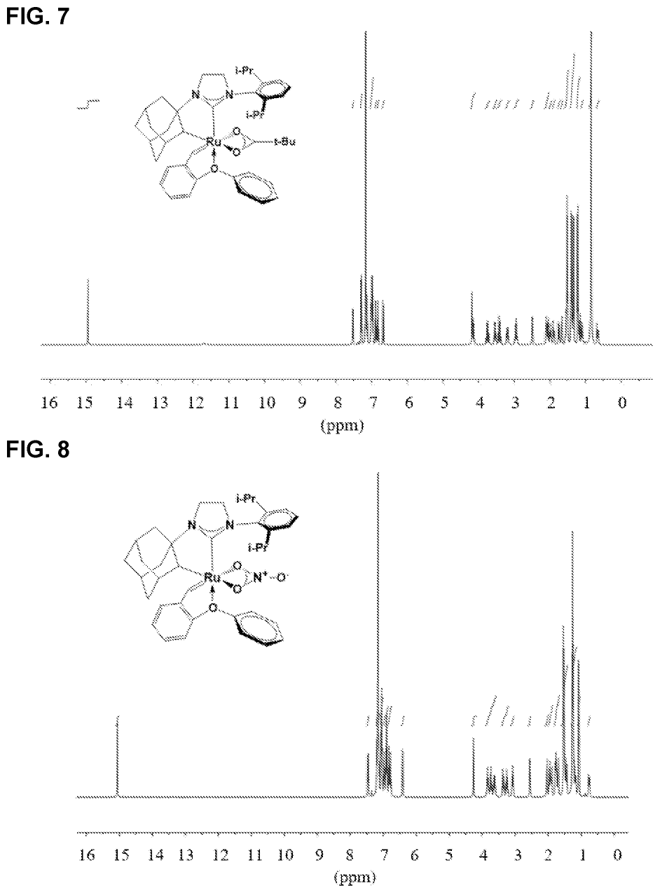

Starting from the corresponding First-Generation Grubbs ruthenium olefin metathesis catalyst, Ru-7 is synthesized in 43% overall yield with >99% purity in four steps, while Ru-8 is synthesized in 30% overall yield with >99% purity in five steps. With the new approach, the key pivalate exchange and the C—H bond activation steps occur smoothly with a 64% yield over two steps. In the first reaction, the anion exchange reached full conversion after treating the ruthenium dichloride precursor with three equivalent of potassium tert-butoxide, furnishing the ruthenium bis-tert-butyl oxide (Ru-6) in 95% yield. In the second reaction, treating Ru-6 with pivalic acid at −20° C. generated Ru-7 in 67% yield upon purification. Further treatment of Ru-7 with 12 equivalents of NH₄NO₃ in THF at room temperature furnished Ru-8 in 70% yield. The detailed procedure for the key steps is similar to that described in Example 3. Representative ¹H-NMR spectra of Ru-7 and Ru-8 as shown in FIGS. 7 and 8, respectively.

### Example 4

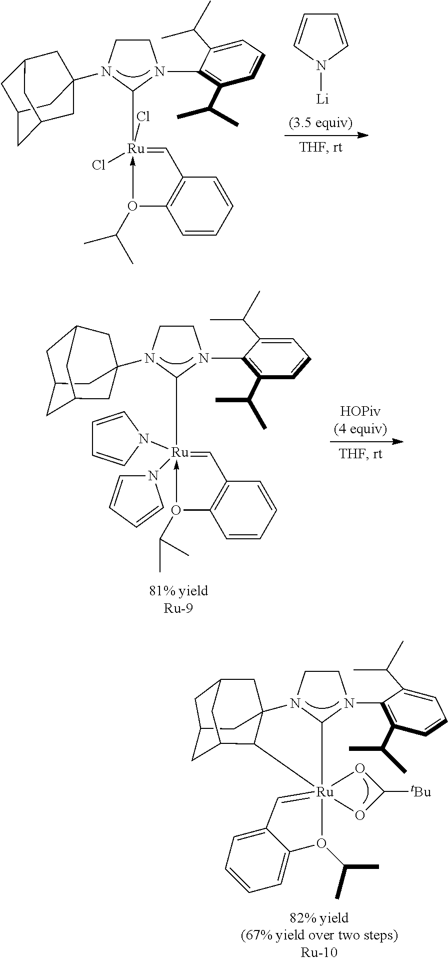

This procedure can also be used to significantly improve the synthesis of Ru-10, which itself is a Z-selective olefin metathesis catalyst and also a precursor for the commercialized Ru-2. In the first reaction, the anion exchange reached full conversion by treating the ruthenium dichloride precursor with 3.5 equivalents of lithium pyrrolate in THF at room temperature, leading to the generation of ruthenium bis-pyrrolate (Ru-9) in 82% yield. In the second reaction, treating Ru-9 with pivalic acid at room temperature led to the formation of Ru-10 in 85% yield. The overall yield of this sequence is 70%, which is significantly higher than the previous route. The detailed experimental procedure is similar to that described in Example 1. The lithium pyrrolate is prepared through the following procedure: An oven-dried flask was fitted with a stir bar and rubber septum and cycled under Ar. To this flask was added dry hexanes (6 mL) and pyrrole (248 mg, 2.7 mmol, 1.0 equiv) through the septum. The solution was cooled to −78° C., with a dry ice/acetone bath, and n-BuLi (1.5 mL, 2.45M in hexane, 3.6 mmol, 0.98 equiv) was added dropwise. The reaction was left to stir for 15 min and the cold bath removed. After stirring for a further 4 h, the solution was transferred into a glass centrifuge tube in a glovebox and pentane (10 mL) was added. The tube was centrifuged and the supernatant removed. This rinse was repeated three more times and the residual solvent was removed using a high vacuum to yield an off-white powder in >90% yield.

### Example 5

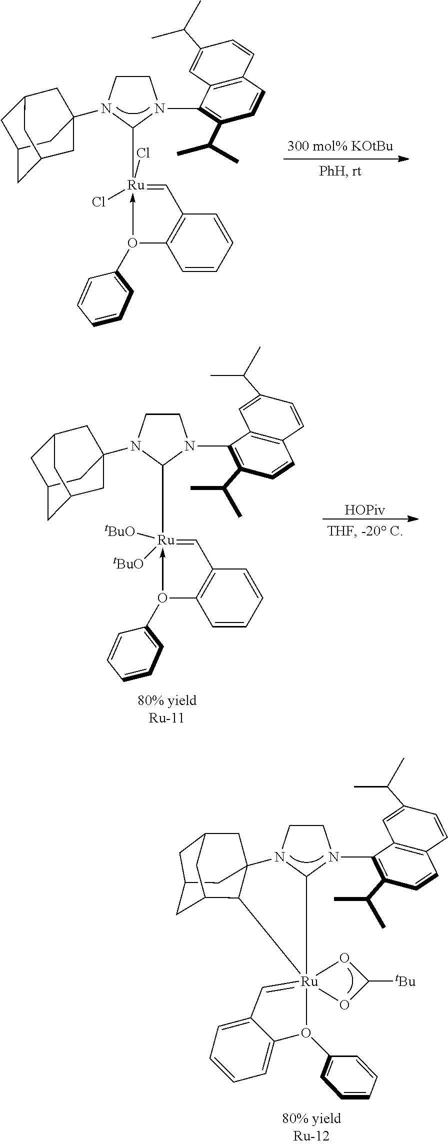

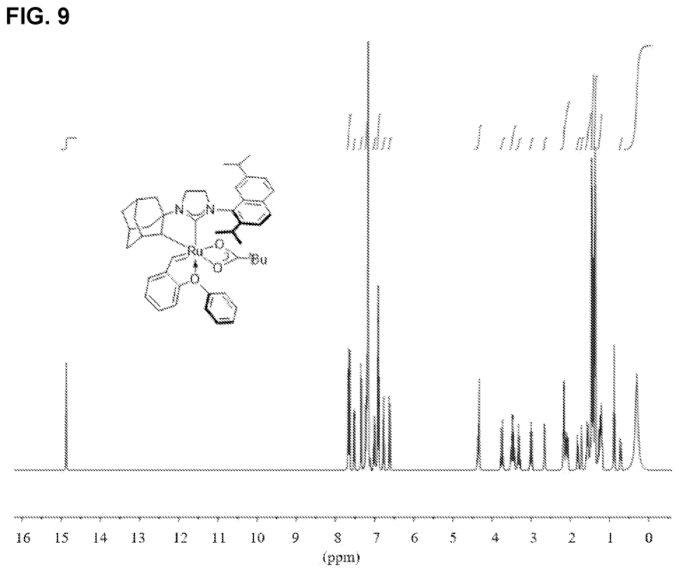

This procedure was also used to synthesize Ru-12, a Z-selective olefin metathesis catalyst bearing a larger NHC ligand than the commercialized Ru-2. In the first reaction, the anion exchange reached full conversion by treating the ruthenium dichloride precursor with 3.0 equivalents of potassium tert-butoxide in benzene at room temperature, leading to the generation of ruthenium bis-tert-butoxide (Ru-11) in 80% yield. In the second reaction, treating Ru-11 with pivalic acid at −20° C. led to the formation of Ru-12 in 80% yield. The detailed experimental procedure is similar to that described in Example 2. A representative ¹H-NMR spectrum of Ru-12 is shown in FIG. 9.

### Example 6

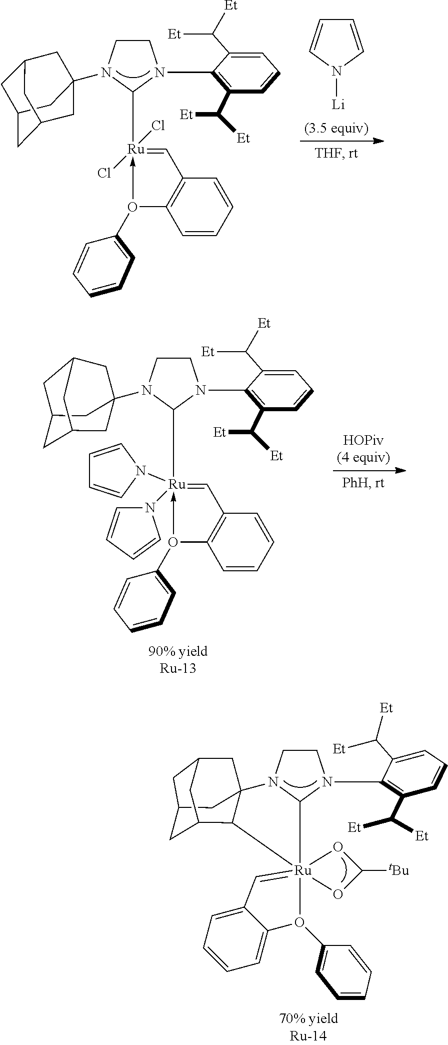

This procedure can also be used to synthesize Ru-14, another Z-selective olefin metathesis catalyst bearing a larger NHC ligand than the commercialized Ru-2. In the first reaction, the anion exchange reached full conversion by treating the ruthenium dichloride precursor with 3.5 equivalents of lithium pyrrolate in THF at room temperature, leading to the generation of ruthenium bis-pyrrolate (Ru-13) in 90% yield. In the second reaction, treating Ru-13 with pivalic acid at room temperature in benzene led to the formation of Ru-14 in 70% yield. The detailed experimental procedure is similar to that described in Example 2. The lithium pyrrolate is prepared by following the procedure described in Example 4.

### Example 7: One Pot Syntheses of Selected Compounds from the Dichloro Compounds

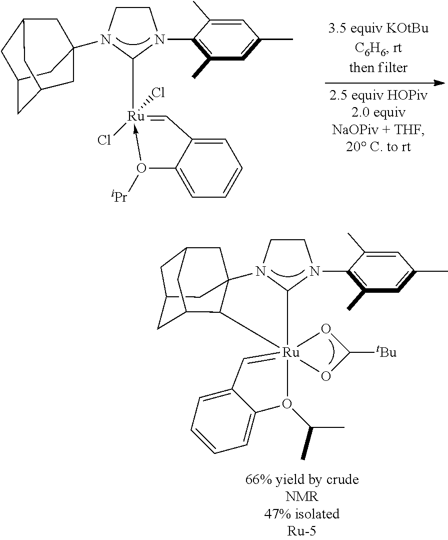

The disclosed methods also include one-pot procedures for the reactions represented herein. Note that in some cases a corresponding alkali metal carboxylate is also needed to ensure full conversion of the starting material or the intermediates.

As represented in the scheme above, this one-pot procedure was applied to prepare Ru-5 under the conditions set forth above The Ru-5 was formed in 66% yield measured by ¹H-NMR and isolated in 47% yield.

KO-tert-Bu (0.3 mmol, 3.0 equiv, 30.4 mg) was added to a solution of starting ruthenium dichloride (0.1 mmol, 1.0 equiv, 64.3 mg) in benzene (1.4 mL) in a 4 mL vial. The suspension was stirred for 7.5 hours at room temperature and then filtered through a short pad of Celite into a 20 mL vial that was pre-charged with NaOPiv (0.2 mmol, 2.0 equiv, 24.8 mg), 1.5 mL THF was used to wash the Celite pad, which was combined with the benzene filtrate (solution A). Another 4 mL vial was charged with PivOH (25 mg, 0.25 mmol, 2.5 equiv) in THF (1.5 mL, solution B). Both vials were put in the glovebox freezer (−20° C.) for 5 minutes. Solution B was quickly transferred into solution A by using a 3 mL syringe, and the resulting mixture was put back into the freezer and sat without stirring for 1.5 hours. The mixture was then taken out and stirred overnight at room temperature before concentrated in vacuo. The residue was transferred onto a short Celite pad. The Celite pad was flushed with pentane (3 mL) and MeOH (1.25 mL) to remove the brownish color band and then benzene (4.5 mL) to obtain the product as a purple solution, Removal of the solvent furnished Ru-5 as a purple solid (31.4 mg, 47% yield).

### Example 8: Metathesis Reactions

Example 8.1: The tolerance of these catalysts toward certain functional groups and the effect of the steric bulk in the R¹⁰ position, relative to existing catalysts, is shown in a series of experiments. A representative catalyst (Ru-5) was shown to efficiently catalyze the cross-metathesis between tert-butyl acrylate and 1-dodecene. The product is generated in 53% yield with >50:1 Z:E selectivity within 2 hours and ultimately in 80% yield with 40:1 Z:E selectivity. In sharp contrast, the best existing catalyst prior to those set forth in this disclosure Ru-2) could only generate the product in 46% yield after 26 hours of reaction.

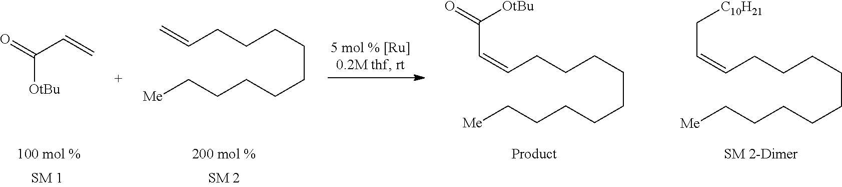

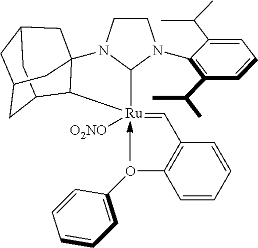

Ru-8

| time | Product | SM 1 | SM 2 | SM 2-Dimer | [Ru] remains | Z/E |
|---|---|---|---|---|---|---|
| 2F | 53% | 47% | 75% | 40% | 80% | — |
| 15h | 77% | 20% | 30% | 50% | 60% | 50:1 by NMR |
| 22h | 80% | 16% | 25% | 53% | 57% | 40:1 by NMR |

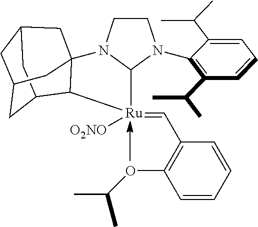

Ru-2 (as benchmark)

| time | Product | SM 1 | SM 2 | SM 2-Dimer | [Ru] remains | Z/E |
|---|---|---|---|---|---|---|
| 2h | 11% | 88% | 150% | 22% | 89% | — |
| 6h | 24% | 75% | 100% | 38% | 86% | — |
| 19h | 40% | 55% | 55% | 50% | 77% | >50:1 by NMR |
| 26h | 46% | 45% | 40% | 55% | 74% | >50:1 by NMR |

Example 8.2: In a second series of experiments, a representative catalyst (Ru-7) was shown to efficiently catalyzed the cross-metathesis between N-benzyl acrylamide and 1-dodecene. The product was generated in 78% yield with >96:4 Z:E selectivity in 16 hours. The best existing catalyst prior to those set forth in this disclosure (Ru-2) could only generate the product in 40% yield after 2 days of reaction.

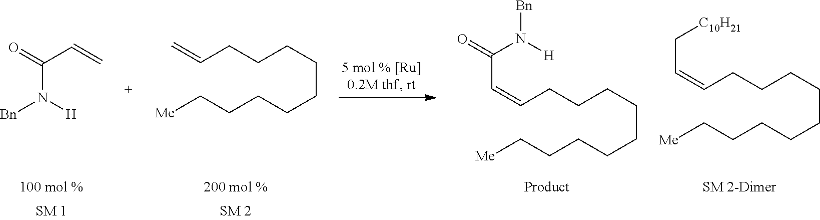

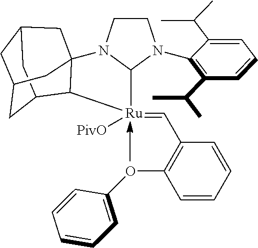

Ru-7

| time | Product | SM 1 | SM 2 | SM 2-Dimer | [Ru] remains | ZE |
|---|---|---|---|---|---|---|
| 2h | 53% | 41% | 90% | 40% | 56% | — |
| 4h | 70% | 26% | 56% | 54% | 30% | — |
| 16h | 78% | 10% | 21% | 62% | 9% | >96:4 by NMR |
| 24h | 77% | 10% | 20% | 62% | 5% | ca 95:5 by NMR |

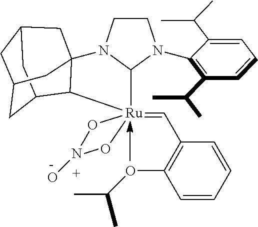

Ru-2

| time | Product | SM 1 | SM 2 | SM 2 - Dimer | [Ru] remains | Z/E |
|---|---|---|---|---|---|---|
| 2h | 4-5% | 95% | 160% | 20% | 85% | — |
| 4h | 9% | 90% | 96% | 50% | 81% | — |
| 16h | 29% | 66% | 50% | 69% | 68% | — |
| 24h | 32% | 62% | 48% | 70% | 66% | >98:2 by NMR |
| 48h | 40% | 54% | 27% | 75% | 58% | >98:2 by NMR |

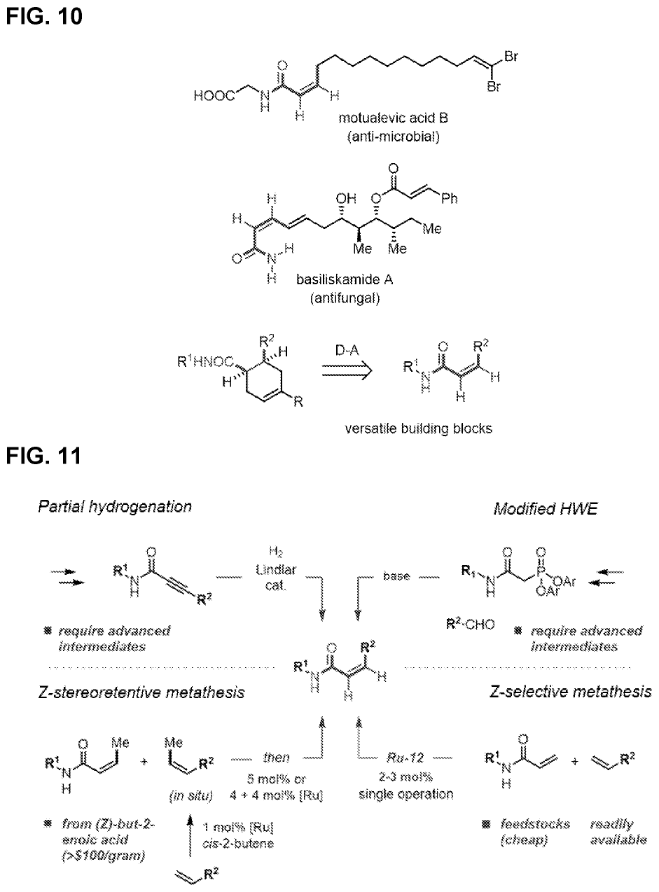

Example 8.3: The inventors have discovered that some of the new ruthenium catalysts made through the new approach, have further shown increased catalytic activity in both selectivity and reactivity, compared to the existing Z-selective olefin metathesis catalysts. For example, the Z-selective cross-metathesis between cheap acrylamides and regular terminal olefins represents a highly desirable approach to construct (Z)-α,β-unsaturated amides (a common motif in bio-active molecules and building blocks, see FIG. 10), as other common alternative approaches generally require the use of advanced synthetic intermediates, expensive starting materials, or harsh reaction conditions (e.g., FIG. 11). Through a series of controlled experiments, the inventors have identified that Ru-12, whose synthesis is enabled by the methods disclosed herein, demonstrates significantly higher reactivity and higher selectivity toward cross-metathesis compared to the commercial catalyst Ru-2:

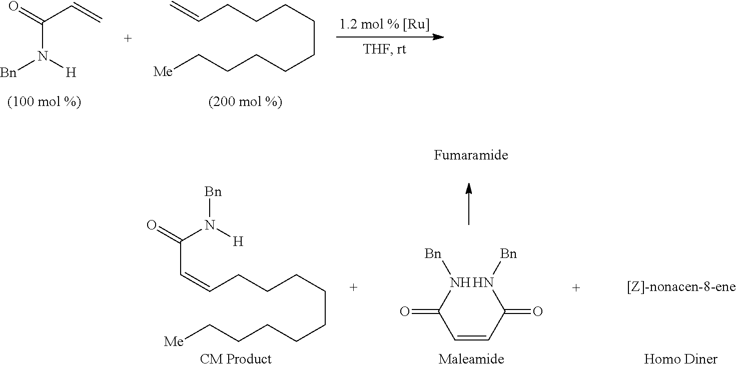 [SMILES: C=CC(=O)NCc1ccccc1 ; C=CCCCCCCCCCC ; CCCCCCCCCC/C=C\C(=O)NCc1ccccc1 ; O=C(/C=C\C(=O)NCc1ccccc1)NCc1ccccc1]

| | | | | Ratio CM |
|---|---|---|---|---|
| | Yield (%) of | Yield (%) of | Yield (%) of | product/ |
| | desired CM | fumaramide/ | nonadec-8- | nonacen-8- |
| Catalyst | product | maleamide | ene | ene |
| 103 | 23 | 1 | 53 | 1:2.3 |
| 104 | 52 | 2.5 | 8 | 6.5:1 |

Experiment 2:

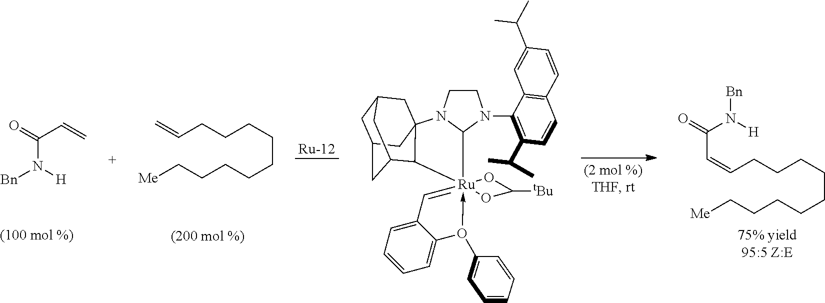

Each patent, patent application, and publication cited or described in this disclosure is hereby incorporated herein by reference, each in its entirety, for all purposes, or at least for the purposes or in the context where it was cited or referenced.

1.-18. (canceled)

19. A method of preparing a compound of Formula (III) or (XIII), the method comprising contacting a compound of Formula (II) or (XII), respectively, or geometric isomer thereof with a carboxylic acid of formula Z—H, where Z—H is C(R¹⁸)(R¹⁹)(R²⁰)COOH, wherein the contacting results in the formation of the compound of Formula (IV) or (XIV), respectively, or geometric isomer thereof: 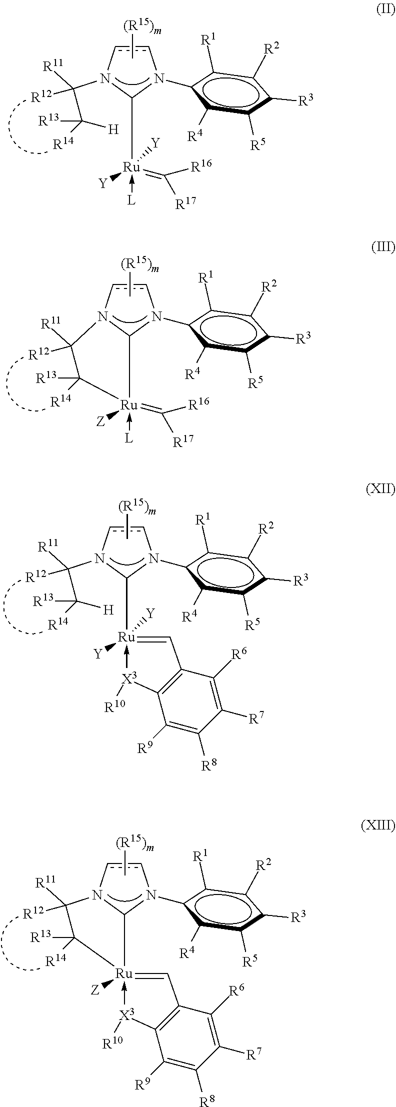
wherein:
R¹, R², R³, R⁴, R⁵, R⁶, R⁷, R⁸, and R⁹ independently are or include hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups;
and/or one or more of R¹ and R² or R² and R³ or R³ and R⁵ or R⁴ and R⁵ or R⁶ and R⁷ or R⁷ and R⁸ or R⁸ and R⁹, together with the carbons to which they are bound, form at least one optionally substituted 5- to 7-membered ring structure;
X³ is —O—, —S—, or —N(R^{10A})—;
R¹⁰ and R^{10A} independently are or include optionally substituted linear, branched, cyclic, bicyclic, or polycyclic C₃₋₂₄ alkyl, optionally substituted aryl, or optionally substituted heteroaryl;
R¹¹, R¹², R¹³, and R¹⁴ independently are or include hydrogen, halogen, optionally substituted alkyl, optionally substituted alkenyl, optionally substituted alkoxyl, optionally substituted aryl, optionally substituted heteroaryl; and/or
one or more pairs of R¹¹, R¹², R¹³, and R¹⁴ are independently optionally linked together to form a cyclic or polycyclic structure;
the designation [symbol] refers to the presence of an optional double bond;
m is 0, 1, 2, 3, or 4;
R¹⁵ independently is or includes an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure;
Y independently is or includes a basic anion coordinated as a ligand to the ruthenium center;
R¹⁶ and R¹⁷ independently are or include hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy;
Z is a carboxylate, optionally of formula C(R¹⁸)(R¹⁹)(R²⁰)C(═O)O⁻;
wherein R¹⁸, R¹⁹, and R²⁰ independently are or include hydrogen, optionally substituted alkyl, optionally substituted alkenyl, optionally substituted aryl, or optionally substituted heteroaryl, provided that not all of R¹⁸, R¹⁹, and R²⁰ are hydrogen; and/or
two or three of R¹⁸, R¹⁹, and R²⁰ are linked together to form a cyclic or polycyclic structure; and
L is a donor ligand, optionally a neutral electron donor ligand, optionally a neutral electron donor solvent or an electron donor ligand linked to R¹⁷.

20. The method of claim 19, further comprising contacting a compound of Formula (I) or (XI), or geometric isomer thereof, with a salt of formula [M⁺][Y⁻], where the anion Y⁻ corresponds to the Y ligand as set forth at least in Embodiment 1, wherein the contacting results in the formation of the compound of Formula (II) or (XII), respectively, or geometric isomer thereof: 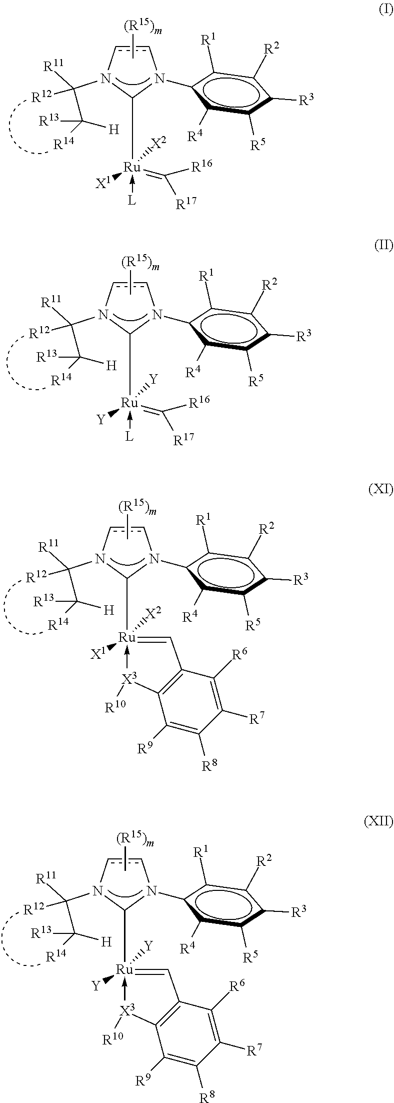
wherein:
X¹ and X² are independently chloro, bromo, or iodo;
M⁺ is an alkali metal cation, an ammonium cation, or other nitrogen-based cation.

21. The method of claim 19, further comprising reacting the compound of formula (III) or (XIII), or geometric isomer thereof, with a trifluoroacetate, nitrate, nitrite, oxalate, phosphate, sulfite, or sulfonate or other anion of a strong acid (H-Q), capable of coordinating the ruthenium as a bidentate ligand, to form a compound of formula (IV) or (XIV), respectively, or geometric isomer thereof: 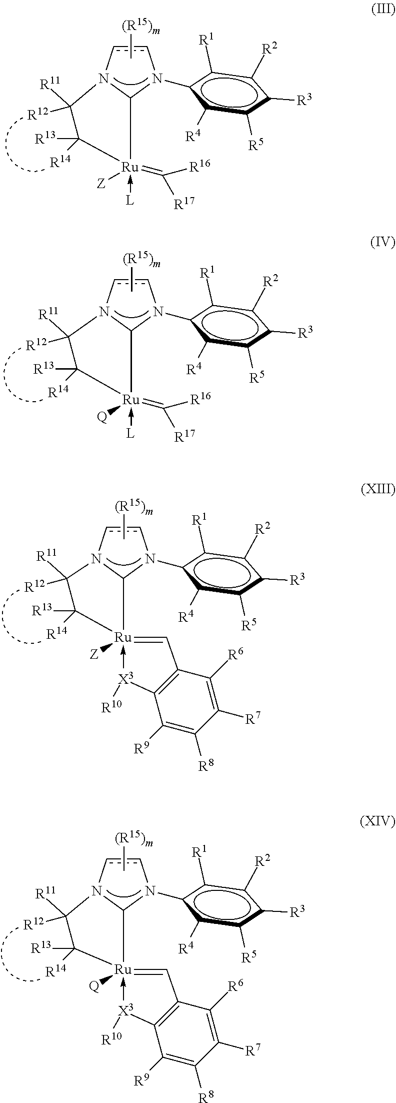
wherein Q is a coordinated trifluoroacetate, nitrate, nitrite, oxalate, phosphate, sulfite, sulfonate, or other anion of a strong acid (H-Q), capable of coordinating the ruthenium as a bidentate ligand.

22. The method of claim 20, wherein the step of contacting the compound of Formula (I) or (XI) with the salt of formula [M⁺][Y⁻], thereby resulting in the formation of the compound of Formula (II) or (XII), respectively, and the subsequent step of contacting the compound of Formula (II) or (XII), respectively, with a carboxylic acid of formula Z—H, thereby resulting in the formation of the compound of Formula (III) or (XIII) are conducted without the isolation of the compound of Formula (II) or (XII), respectively.

23. The method of claim 19, wherein R¹ and R⁴ are optionally substituted C₁₋₆ alkyl and R², R³, and R⁵ are independently hydrogen or optionally substituted C₁₋₆ alkyl.

24. The method of claim 19, wherein R¹, R⁴, and optionally R³ are independently branched C₃₋₆ alkyl, branched C₄₋₆ alkyl, or phenyl.

25. The method of claim 19, wherein R¹ and R⁴ are optionally substituted branched C₃₋₆ alkyl, branched C₄₋₆ alkyl, or phenyl, and R², R³, and R⁵ are independently hydrogen or optionally substituted C₁₋₆ alkyl

26. The method of claim 19, wherein 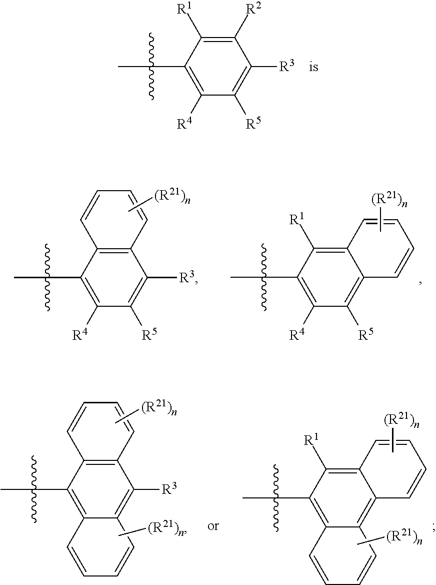
wherein R²¹ is halogen, optionally substituted C₁₋₁₂ alkyl, optionally substituted C₂₋₁₂ alkenyl, optionally substituted C₁₋₁₂ alkoxy, optionally substituted C₆₋₂₄-aryl, optionally substituted C₃₋₂₄-heteroaryl, or nitro; and
n is independently 0 to 4 at each occurrence.

27. The method of claim 19, wherein 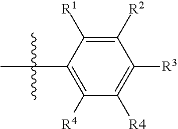
is:

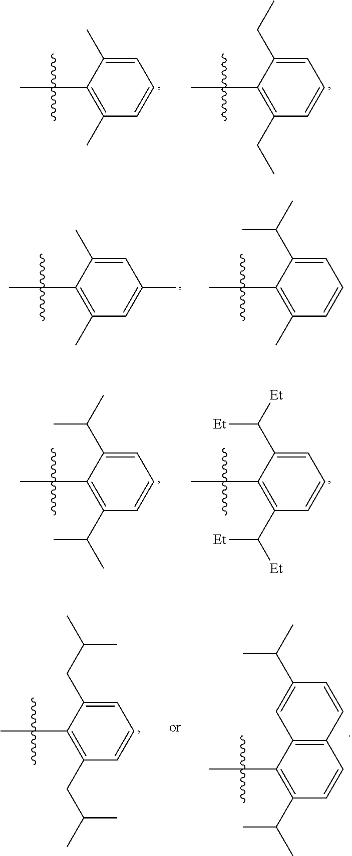

28. The method of claim 19, wherein the X³ is —O—.

29. The method of claim 19, wherein R¹⁰ and/or R^{10A} is substituted or unsubstituted phenyl or a linear, branched, cyclic, bicyclic, or polycyclic C₃₋₂₄ alkyl.

30. The method of claim 19, wherein 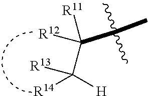
comprises an optionally alkyl substituted adamantyl moiety.

31. The method of claim 19, wherein:
(a) the compound of Formula (II) is the compound of Formula (II-A) and the compound of Formula (III) is the compound of Formula (III-A): 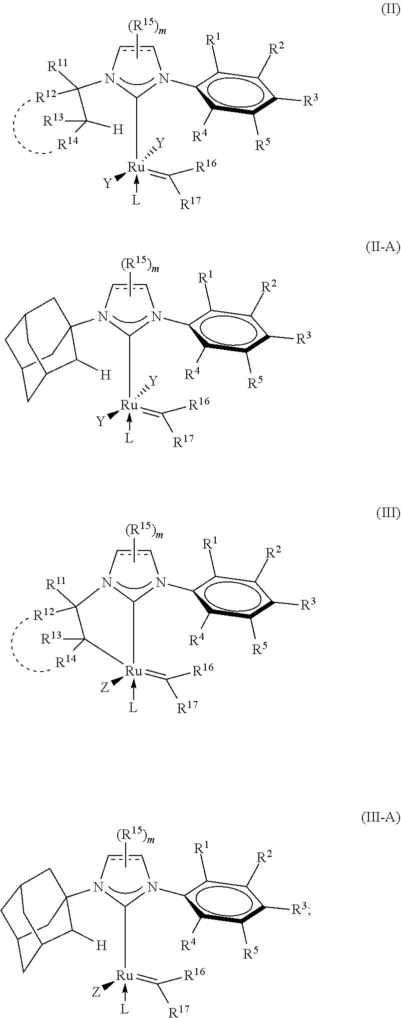
and
(b) the compound of Formula (XII) is the compound of Formula (XII-A) and the compound of Formula (XIII) is the compound of Formula (XIII-A):

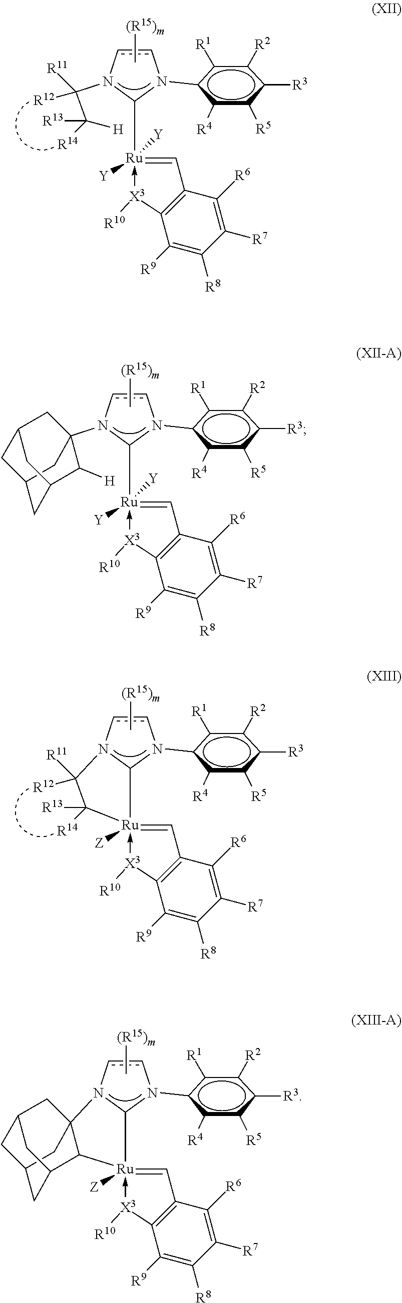

32. The method of claim 20, wherein:
(a) the compound of Formula (I) is the compound of Formula (I-A): 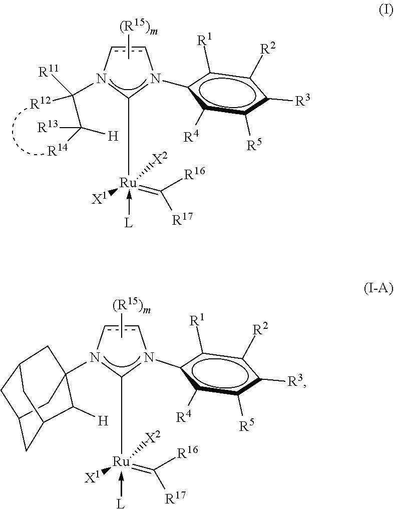
or
(b) the compound of Formula (XI) is the compound of Formula (XI-A):

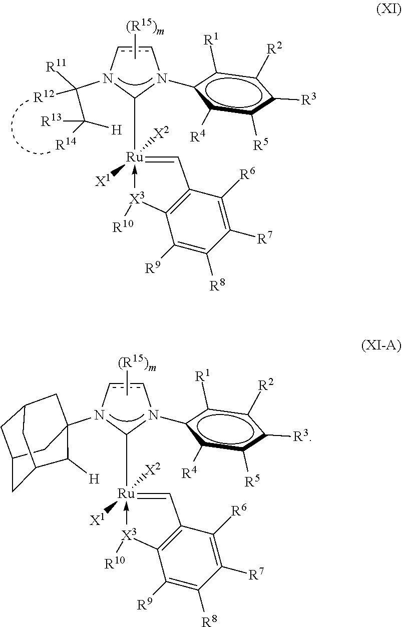

33. The method of claim 21, wherein:
(a) the compound of Formula (IV) is the compound of Formula (IV-A): 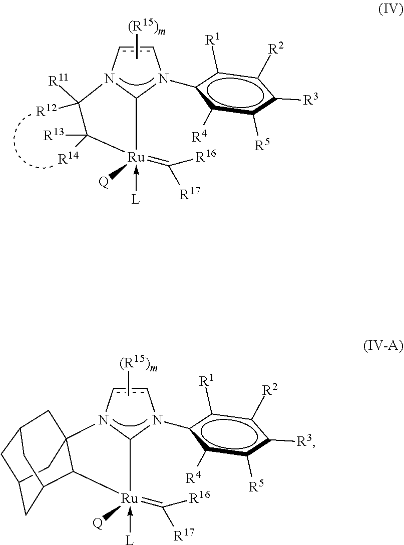
and
(b) the compound of Formula (XIV) is the compound of Formula (XIV-A):

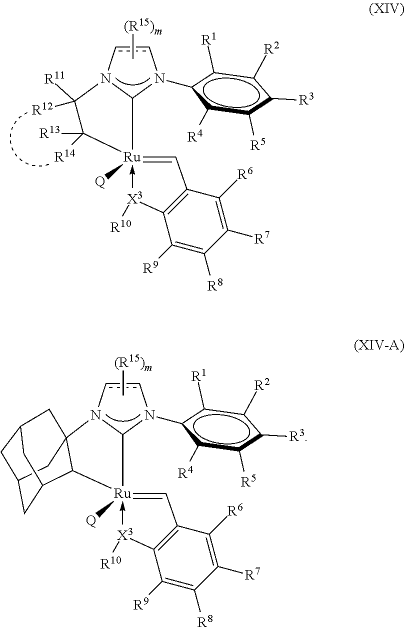

34. A ruthenium compound of Formula (XX-A), (XX-B), (XX-C) or (XX-D): 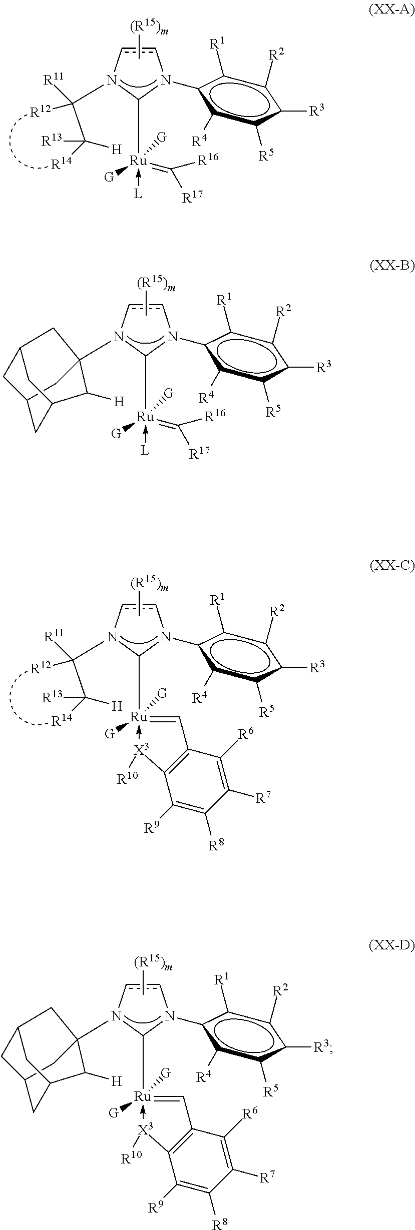
wherein:
R¹, R², R³, R⁴, R⁵, R⁶, R⁷, R⁸, and R⁹ are independently hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups;
and/or one or more of R¹ and R² or R² and R³ or R⁶ and R⁷ or R⁷ and R⁸ or R⁸ and R⁹, together with the carbons to which they are bound, form at least one optionally substituted 5- to 7-membered ring structure;
X³ is —O—, —S—, or —N(R^{10A})—,
R¹⁰ and R^{10A} are optionally substituted linear, branched, cyclic, bicyclic, or polycyclic C₃₋₂₄ alkyl, optionally substituted aryl, or optionally substituted heteroaryl;
R¹¹, R¹², R¹³, and R¹⁴ are independently hydrogen, halogen, optionally substituted alkyl, optionally substituted alkenyl, optionally substituted alkoxyl, optionally substituted aryl, optionally substituted heteroaryl; and/or
one or more pairs of R¹¹, R¹², R¹³, and R¹⁴ are independently optionally linked together to form a cyclic or polycyclic structure;
the designation [symbol] refers to the presence of an optional double bond;
m is 0, 1, 2, 3, or 4;
R¹⁵ is independently an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure;
R¹⁶ and R¹⁷ are independently hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy; G is independently:
(a) chloro, bromo, or iodo, or a combination thereof;
(b) a basic anion coordinated as a ligand to the ruthenium center, each basic anion comprising hydroxide, C₁₋₁₂ alkoxide, amidate anion, substituted or unsubstituted phenoxide, substituted or unsubstituted pyrrolate, substituted or unsubstituted indolate, substituted or unsubstituted isoindolate, or substituted or unsubstituted imidazolate; and
L is a neutral electron donor ligand, optionally a neutral electron donor solvent or an electron donor ligand linked to R¹⁷, with the proviso that:
when G is a chloro, bromo, or iodo, or a combination thereof, then
(i) R¹ and R⁴ are independently optionally substituted C₄₋₁₂, C₅₋₁₂, C₆₋₁₂, C₇₋₁₂, or C₈₋₁₂ alkyl, optionally substituted aryl, optionally substituted heteroaryl, or optionally substituted heteroaryloxy; and/or one or more of R¹ and R² or R² and R³ or R³ and R⁵ or R⁴ and R⁵, together with the carbons to which they are bound, form at least one optionally substituted 5- to 7-membered ring structure;
(ii) R¹ and R⁴ are independently optionally substituted branched C₄₋₁₂, C₅₋₁₂, C₆₋₁₂, C₇₋₁₂, or C₈₋₁₂ alkyl; or
(iii) neither R¹⁰ nor R^{10A} are isopropyl or optionally substituted phenyl.

35. A ruthenium compound of Formula (Y-A), (Y-B), (Y-C), or (Y-D): 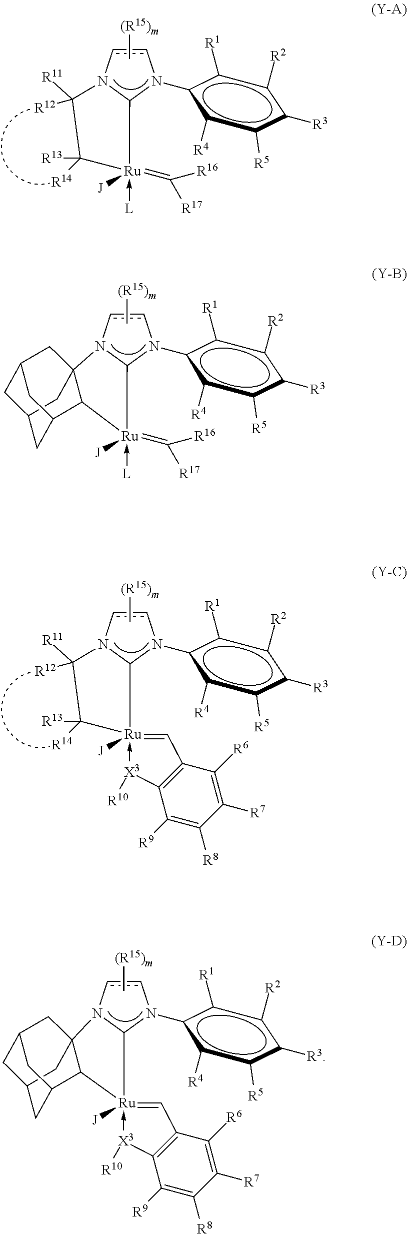
wherein:
R¹, R², R³, R⁴, R⁵, R⁶, R⁷, R⁸, and R⁹ are independently hydrogen, halogen, optionally substituted alkyl, optionally substituted alkenyl, optionally substituted alkoxy, optionally substituted aryl, optionally substituted aryloxy, optionally substituted heteroaryl, optionally substituted heteroaryloxy, or nitro groups;
and/or one or more of R¹ and R² or R² and R³ or R⁶ and R⁷ or R⁷ and R⁸ or R⁸ and R⁹, together with the carbons to which they are bound, form at least one optionally substituted 5- to 7-membered ring structure;
X³ is —O—, —S—, or —N(R^{10A})—;
R¹⁰ and R^{10A} are optionally substituted linear, branched, cyclic, bicyclic, or polycyclic C₃₋₂₄ alkyl, optionally substituted aryl, or optionally substituted heteroaryl;
R¹¹, R¹², R¹³, and R¹⁴ are independently hydrogen, halogen, optionally substituted alkyl, optionally substituted alkenyl, optionally substituted alkoxyl, optionally substituted aryl, optionally substituted heteroaryl; and/or
one or more pairs of R¹¹, R¹², R¹³, and R¹⁴ are independently optionally linked together to form a cyclic or polycyclic structure;
the designation [symbol] refers to the presence of an optional double bond;
m is 0, 1, 2, 3, or 4;
R¹⁵ is independently an optionally substituted alkyl, optionally substituted alkenyl, optionally substituted alkoxy, optionally substituted aryl, optionally substituted aryloxy, optionally substituted heteroaryl, optionally substituted heteroaryloxy, halo, cyano, nitro, or any two of R¹⁵ may be linked to form a cyclic structure;
J is independently:
(a) a monodentate ligand, such as those set forth elsewhere herein for X¹ or Y, for example a phenoxide substituted at least in its 2,6-positions;
(b) a carboxylate [optionally of formula C(R¹⁸)(R¹⁹)(R²⁰)C(═O)O⁻], wherein R¹⁸, R¹⁹, and R²⁰ are independently hydrogen, optionally substituted alkyl, optionally substituted alkenyl, optionally substituted aryl, or optionally substituted heteroaryl, provided that not all of R¹⁸, R¹⁹, and R²⁰ are hydrogen; and/or two or three of R¹⁸, R¹⁹, and R²⁰ are linked together to form a cyclic or polycyclic structure; or
(c) a trifluoroacetate, nitrate, nitrite, oxalate, phosphate, sulfite, sulfonate, or other anion of a strong acid (H-Q), capable of coordinating the ruthenium as a bidentate ligand;
R¹⁶ and R¹⁷ are independently hydrogen, halogen, optionally substituted alkyl, optionally substituted alkenyl, optionally substituted alkoxy, optionally substituted alkenyl, optionally substituted aryl, optionally substituted aryloxy, optionally substituted heteroaryl, or optionally substituted heteroaryloxy; and
L is a neutral electron donor ligand, optionally a neutral electron donor solvent or an electron donor ligand linked to R¹⁷;
with the proviso that when J is the substituted phenoxide, the carboxylate of formula C(R¹⁸)(R¹⁹)(R²⁰)C(═O)O⁻, or the trifluoroacetate, the nitrate, the nitrite, the oxalate, the phosphate, the sulfite, the sulfonate, or the other anion of a strong acid (H-Q), capable of coordinating the ruthenium as a bidentate ligand, then
(i) R¹ and R⁴ are independently optionally substituted C₄₋₁₂ alkyl, optionally substituted aryl, optionally substituted heteroaryl, or optionally substituted heteroaryloxy; and/or one or more of R¹ and R² or R² and R³ or R³ and R⁵ or R⁴ and R⁵, together with the carbons to which they are bound, form at least one optionally substituted 5- to 7-membered ring structure, or
(ii) R¹⁰ is optionally substituted linear, branched, cyclic, bicyclic, or polycyclic C₄₋₁₂ alkyl, optionally substituted aryl, optionally substituted heteroaryl, or optionally substituted heteroaryloxy.

36. A process comprising contacting at least one alkene, alkyne, or enyne with the ruthenium compound of claim 35, the contacting resulting in a metathesis reaction comprising:
(a) a cross metathesis (CM) of two different alkenes, or an alkene, alkyne, and/or enyne;
(b) a ring closing metathesis of a diene (RCM) leading to the formation of cycloalkene ring;
(c) a homodimerization of two olefins;
(d) an acyclic diene metathesis (ADMET) leading to the formation of an oligomer and/or a polymer;
(e) a ring opening cross metathesis (ROCM) leading to an acyclic diene;
(f) a ring opening metathesis polymerization (ROMP) of a cycloalkene leading to a polymer; or
(g) ethenolysis of an E/Z isomer mixture of olefins to provide an enrichment of the E-olefin content of the mixture.

37. The process of claim 36, wherein a product of the metathesis reaction is predominantly a Z-isomer olefin.
